(19) United States
(12) Patent Application Publication
(10) Pub. No.: US 2023/0270856 A1
(43) Pub. Date: Aug. 31, 2023

(54) FUSION PROTEIN AND USE THEREOF

(71) Applicant: SUPERMAB (BEIJING) BIOTECH CO., LTD., Beijing (CN)

(72) Inventor: Yu LIU, Beijing (CN)

(21) Appl. No.: 18/006,591

(22) PCT Filed: May 19, 2021
(86) PCT No.: PCT/CN2021/094655

§ 371 Date: Jan. 24, 2023

(30) Foreign Application Priority Data
Jul. 24, 2020 (CN) 202010720984.6

(51) Int. Cl. A61K 39/00; C07K 14/55; C07K 16/00; C07K 7/06

(52) U.S. Cl. CPC A61K 39/4611; C07K 14/55; C07K 16/00; C07K 7/06; A61K 39/4613; A61K 39/4633; A61K 39/4635; C07K 2319/30; C07K 2317/622

(57) ABSTRACT

The present invention relates to a fusion protein and the use thereof. The fusion protein includes, from N to C terminals, a first moiety, an Fc segment, a linking moiety comprising a moiety selected from a linker and a protein or polypeptide selected from IL2 or scFv, and a substrate moiety of transpeptidase A; the linker includes a sequence selected from the group consisting of (1) (GGGGS)n, wherein when the linking moiety includes the protein or polypeptide and the linker, n≥1; when the linking moiety only includes the linker, n≥3; and (2) (EAAAK)n, n≥1; the substrate moiety includes LPXTG. The fusion protein can be directly connected to cells to enable the cells to have a targeting property, is more simple than an existing method for preparing targeting cells by means of cell transfection, and can also reduce the risk possibly generated by an effector cell genome operation.

The present application claims priority to the Chinese Patent Application, titled “FUSION PROTEIN AND USE THEREOF” and filed on Jul. 24, 2020, the content of which is herein incorporated by reference in its entirety.

### TECHNICAL FIELD

The present invention relates to the field of proteins, and specifically to fusion proteins suitable for connecting antibody Fc regions to cells.

### BACKGROUND TECHNOLOGY

Cell therapy, a new technology for disease treatment emerging in recent years, refers to the cells with enhanced functions produced after a particular treatment utilizing the properties of certain cells with specific functions (such as stem cells and immune cells), which are then infused into the body for the purpose of treating diseases. With the continuous development of basic theories, technical means and clinical medical exploration study of stem cell therapy, immune cell therapy and gene editing, cell therapy products have provided new treatment ideas and methods for some severe and refractory diseases, showing increasingly high application value.

Targeted therapy is a drug therapy to treat diseases by interfering with specific molecules. Targeted cell therapy generated by combining targeted therapy with cell therapy, such as CAR-T cells (chimeric antigen receptor T cells), TCR-T cells (T cell receptor T cells), CAR-NK cells (chimeric antigen receptor NK cells), makes cells with targeting by expressing the antibody scFv segments on the cell surface. Targeted cell therapy has shown excellent clinical efficacy in the treatment of malignant diseases such as tumors, and has been shown to be a very promising strategy for disease treatment. In 2018, two CAR-T drugs have been approved by the US FDA for marketing. However, for existing cell therapies, cell preparation cycles and cost issues directly lead to significant limitations in the timeliness and breadth of cell therapy (Cornetta K., Pollok K. E., Miller A. D. Transduction of Primary Hematopoietic Cells by Retroviral Vectors. Cold Spring Harbor Protocols 2008, 2008, 4884.10.1101; Shearer R. F., Saunders D. N. Experimental Design for Stable Genetic Manipulation in Mammalian Cell Lines: Lentivirus and Alternatives. Genes Cells 2015, 20, 1-10. 10.1111).

Antibodies, also known as immunoglobulins (Ig for short), can be classified into five classes: IgG, IgM, IgA, IgE, and IgD, according to their physicochemical properties and biological functions. The Fab (fragment of antigen binding) segment of an antibody is an antigen-binding fragment, composed of a complete light chain and a VH and a CH1 domain of a heavy chain; an Fc segment is a fragment crystallizable (Fc), which region is composed of two or three constant domains of the heavy chain according to the type of the antibody. For example, an Fc domain of IgG comprises CH2 and CH3 domains of a heavy chain. The Fc segment can bind various Fc receptors (FcRs) and other immune molecules, a process that triggers different target cell killing effects, including antibody-dependent cell-mediated cytotoxicity (ADCC), antibody-dependent cellular phagocytosis (ADCP), and complement-dependent cytotoxicity (CDC) (Woof J, Burton D. Human antibody-Fc receptor interactions illuminated by crystal structures. Nat Rev Immunol. 2004, 4 (2): 89-99. Heyman B. Complement and Fc-receptors in regulation of the antibody response. Immunol Lett. 1996, 54 (2-3): 195-199.), and is the key effector segment of a therapeutic antibody to exert its utility in vivo (Seidel U J, Schlegel P, Lang P. Natural killer cell mediated antibody-dependent cellular cytotoxicity in tumor immunotherapy with therapeutic antibodies; Hatjiharissi, E, Xu, L, Santos, D et al. Increased natural killer cell expression of CD16, augmented binding and ADCC activity to rituximab among individuals expressing the Fc RIIIa-158 V/V and V/F polymorphism. Blood 2007; 110: 2561-4; Musolino, A, Naldi, N, Bortesi, B et al. Immunoglobulin G fragment C receptor polymorphisms and clinical efficacy of trastuzumab-based therapy in patients with HER-2/neu-positive metastatic breast cancer. J Clin Oncol 2008; 26: 1789-96; Lo Nigro C, Macagno M, Sangiolo D, Bertolaccini L, Aglietta M, Merlano M C. NK-mediated antibody-dependent cell-mediated cytotoxicity in solid tumors: biological evidence and clinical perspectives. Ann Transl Med. 2019 March; 7(5):105). The Fc fusion protein refers to the fusion of the Fc segment of an antibody with a protein molecule with a specific biological function using genetic engineering techniques, which not only has the original activity of the functional protein, but also has certain properties of the antibody, such as functions of ADCC, CDC and ADCP. It can thus be seen that if an Fc segment-containing protein (such as an antibody or an Fc fusion protein with an antigen-binding region) is directly connected to the surface of an effector cell, it can enable the cell to have targeting for recognition of specific antigens and solve the problems of the complicated preparation of existing therapeutic cells. Furthermore, after this protein is bound to the corresponding antigen, the Fc segment of which can further effectively stimulate the killing activity of the effector cell.

At present, there are a variety of methods to connect various molecules (such as nucleic acids, proteins) to the cell surface, e.g. connecting molecules containing compatible groups to the cell using amino, sulfhydryl or thiol groups on the cell membrane surface (Lee D Y, Park S J, Nam J H, Byun Y. Tissue Engineering. 2006; 12:615-623; Hsiao S C, Shum B J, Onoe H, Douglas E S, Gartner Z J, Mathies R A, et al. Langmuir. 2009; 25:6985-6991; Krishnamachari Y, Pearce M E, Salem A K. Advanced Materials. 2008; 20:989-993; Murciano J C, Medinilla S, Eslin D, Atochina E, Cines D B, Muzykantov V R. Nature Biotechnology. 2003; 21:891-896; Stephan M T, Moon J J, Um S H, Bershteyn A, Irvine D J. Nature Medicine. 2010; 16:1035-1041), anchoring target proteins containing hydrophobic glycosylphosphatidylinositol (GPI) to the cell membrane (Notohamiprodjo M, Djafarzadeh R, Mojaat A, von Luttichau I, Grone H J, Nelson P J. Protein Eng Des Sel. 2006; 19:27-35; Ko I K, Kean T J, Dennis J E. Biomaterials. 2009; 30:3702-3710; Kim S A, Peacock J S. Journal of Immunological Methods. 1993; 158:57-65), adsorbing target molecules to the cell using PEG with cations or nanomaterials (Wilson J T, Krishnamurthy V R, Cui W X, Qu Z, Chaikof E L. Journal of the American Chemical Society. 2009; 131:18228-18229; Stephan M T, Moon J J, Urn S H, Bershteyn A, Irvine D J. Nature Medicine. 2010; 16:1035-1041; Gao H, Shi W, Freund L B. Proc Natl Acad Sci USA. 2005; 102:9469-9474), connecting cell-target molecules using cell surface receptor-ligand (Swiston A J, Cheng C, Um S H, Irvine D J, Cohen R E, Rubner M F. Nano Letters. 2008; 8:4446-4453), in addition to the click chemistry method (Swee L K, Lourido S, Bell G W, Ingram J R, Ploegh H L. One-step enzymatic modification of the cell surface redirects cellular cytotoxicity and parasite tropism. ACS Chem Biol. 2015 Feb. 20; 10(2):460-5; Nikić I, Kang J, Girona G E, Aramburu I V, Lemke E A. Labeling proteins on live mammalian cells using click chemistry. Nat Protoc. 2015 May; 10(5):780-91; Horisawa K. Front Physiol. Specific and quantitative labeling of biomolecules using click chemistry. 2014 Nov. 24; 5:457; Uttamapinant C, Sanchez M I, Liu D S, Yao J Z, Ting A Y. Site-Specific Labeling of Proteins in Live Mammalian Cells using Click Chemistry. Nat Protoc. 2013 August; 8(8):1620-34; Li J, Chen M, Liu Z, Zhang L, Felding B H, Moremen K W, Lauvau G, Abadier M, Ley K, Wu P. A Single-Step Chemoenzymatic Reaction for the Construction of Antibody-Cell Conjugates. ACS Cent Sci. 2018 Dec. 26; 4(12):1633-1641), the Sortase A (SrtA for short) catalytic method (Pishesha N, et al., Engineered Erythrocytes Covalently Linked to Antigenic Peptides Can Protect Against Autoimmune Disease. Proc. Natl. Acad. Sci. U.S.A 2017, 114, 3157-3162; Chen I, Don B M, Liu D R. A general strategy for the evolution of bond-forming enzymes using yeast display. Proc Natl Acad Sci USA. 2011 Jul. 12; 108(28):11399-404; Tanaka T, Yamamoto T, Tsukiji S, Nagamune T. Site-specific protein modification on living cells catalyzed by Sortase. Chembiochem. 2008 Mar. 25; 9(5):802-7; US 2016.0122707A1), and the like.

Among them, the Sortase A proceeds the connecting reaction with a substrate containing oligomeric Gly sequences at the N-terminus by recognizing and cleaving the peptide bond between T/G in the LPXTG substrate sequence (Kruger R G, Otvos B, Frankel B A, et al. Analysis of the substrate specificity of the Staphylococcus aureus sortase transpeptidase SrtA. Biochemistry, 2004, 43(6): 1541-1551; Suree N, Liew C K, Villareal V A, et al. The structure of the Staphylococcus aureus sortase-substrate complex reveals how the universally conserved LPXTG sorting signal is recognized. J Biol Chem, 2009, 284(36): 24465-24477). Although it has been found that Sortase A can connect a variety of molecules (such as biotin, polypeptide, antibody heavy chain variable region VHH fragment) directly to the cell surface (US20160122707A1; Jeong H J, et al., Generation of Ca2+-independent sortase A mutants with enhanced activity for protein and cell surface labeling. PLoS One. 2017 Dec. 4; 12(12):e0189068; Chen I, et al., A general strategy for the evolution of bond-forming enzymes using yeast display. Proc Natl Acad Sci USA. 2011 Jul. 12; 108(28):11399-404), no study has yet reported the successful connection of an intact antibody or Fc fusion protein containing an Fc segment (fragment crystallizable region) directly to the cell surface by Sortase A, due to the large molecular weight and complex structure of the Fc segment.

In view of the fact that connecting an Fc segment-containing protein to a cell not only allows the cell to have targeting, but also allows the Fc segment to further enhance the cytotoxic activity of effector cells, there is a need in the field for methods of connecting an Fc segment-containing protein directly to the cell surface.

### SUMMARY OF THE INVENTION

The purpose of the present invention is to provide an Fc segment-containing protein molecule and a method of connecting the Fc segment-containing protein molecule directly to the cell surface in response to the problems and deficiencies in the prior art. The effector cells obtained by this method can bind to the corresponding soluble antigens and cell surface antigens through the Fc segment-containing proteins connected on their surface, and then the Fc segment of the protein can finally achieve specific antigen clearance and target cell killing by binding to the Fc receptor on the surface of the effector cells and triggering the effector cell activation-related signaling pathways. The effector cells can be used to prevent and treat a disease caused by abnormal cell proliferation and/or function in the body, such as a tumor, an autoimmune disease and an infectious disease.

To address the above technical problems, first provided herein is an Fc segment-containing fusion protein (such as an antibody and an Fc fusion protein) which may be directly connected to the cell surface mediated by a Sortase A. Also provided herein is a method of connecting the Fc segment-containing fusion protein (such as an antibody and an Fc fusion protein) directly to the cell surface by a Sortase A protein.

In one aspect, the present invention provides an Fc segment-containing fusion protein comprising, from N to C terminus, a first moiety, an Fc segment, a linking moiety, and a substrate moiety of Sortase A. The moieties may be connected to each other directly or through one or more amino acid residues.

In one embodiment, the linking moiety comprises a portion or a region selected from a linker or a protein or polypeptide. For example, the linking moiety is a separate linker, a separate protein or polypeptide, or a portion comprising a protein or polypeptide and a linker from the N-terminus to the C-terminus. Preferably, the protein or polypeptide is selected from scFv.

In one embodiment, the linker comprises a sequence selected from the group consisting of:

(1) (GGGGS)n, wherein n≥1 when the linking moiety comprises the protein or polypeptide and the linker, and an integer of n≥3 when the linking moiety comprises only the linker; and

(2) (EAAAK)ₙ, an integer of n≥1.

In one embodiment, the substrate moiety comprises the sequence as shown in LPXTG.

In one embodiment, the first moiety may be selected from F(ab′)₂, F(ab′), Fab, Fv, scFv, receptor, and ligand. The first moiety may also be any other binding partner of the relevant pathogen.

In one embodiment, the Fc segment is a wild-type Fc segment or a variant Fc segment.

In one embodiment, the Fc segment is selected from an Fc segment of IgG, IgM, IgA, IgD, and IgE.

In one embodiment, the Fc segment is selected from an Fc segment of IgG1, IgG2, IgG3, and IgG4.

In one embodiment, the fusion protein comprises the structure of any one of:
full-length antibody-GGGGS-GGGGS-GGGGS-LPETGG;
Full-length antibody-EAAAK-LPETGG;
Full-length antibody-IL2-LPETGG;
Full-length antibody-scFv-GGGGS-LPETGG;
Full-length antibody-scFv-EAAAK-LPETGG;
scFv-Fc segment-GGGGS-GGGGS-GGGGS-LPETGG; or
scFv-Fc segment-EAAAK-LPETGG.

In another aspect, the present invention provides a nucleic acid encoding the fusion protein according to the present invention.

In one embodiment, the nucleic acid comprises the encoding sequence of SEQ ID Nos. 4 and 12, SEQ ID Nos. 4 and 14, SEQ ID Nos. 4 and 16, SEQ ID Nos. 4 and 18, SEQ ID Nos. 4 and 20, SEQ ID No. 28, SEQ ID No. 30, or SEQ ID No.

32.

In another aspect, the present invention provides a vector comprising the nucleic acid according to the present invention.

In another aspect, the present invention provides a host cell comprising the vector according to the present invention.

In another aspect, the present invention provides a method for generating the fusion protein according to the present invention, comprising:

(1) mixing equimolarly a vector expressing a heavy chain and a vector expressing a light chain in the presence of a light chain;

(2) introducing the vector mixture into the host cell and expressing it for a suitable time under conditions suitable for the expression of the fusion protein; and

(3) recovering the medium supernatant and purifying the fusion protein.

In another aspect, the present invention provides a method for connecting the Fc segment-containing fusion protein according to the present invention to the surface of a cell comprising a step of contacting the cell with the Fc segment-containing fusion protein and a Sortase A.

In one embodiment, the cell is an effector cell. In one embodiment, the cell is an NK cell or a T cell. For example, the cell is a peripheral blood NK cell, a peripheral blood T cell, and a cord blood NK cell. For example, the cell is an NK92-FcγRIII cell.

In another aspect, the present invention provides a cell or progeny thereof prepared according to the method of the present invention.

In another aspect, the present invention provides a pharmaceutical composition comprising the cell according to the present invention and a pharmaceutically acceptable carrier.

In another aspect, the present invention provides the use of the cell according to the present invention or the pharmaceutical composition according to the present invention in the manufacture of a medicament used for a disease.

In one embodiment, the disease is a disease caused by abnormal cell proliferation and/or function. For example, the disease is a tumor, an autoimmune disease, and/or an infectious disease.

Herein, the linking moiety comprises IL2 or scFv, wherein the amino acid sequence of IL2 may be GGGGSGSAPTSSSTKKTQLQLEHLLLDLQMILNGINNYKNPKLTRMLTFK FYMPKKATELKHLQCLEEELKPLEEVLNLAQSKNFHLRPRDLISNINVIVL ELKGSETTFMCEYADETATIVEFLNRWITFCQSIISTLTRT (sequence 33); the amino acid sequence of scFv may be

(sequence 34)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKGGGGSGGGGSGGGGSEVQLVESGGGLVQPGGSLRLSCAASGFT
FSDSWIHWVRQAPGKGLEWVAWISPYGGSTYYADSVKGRFTISADTSKNT
AYLQMNSLRAEDTAVYYCARRHWPGGFDYWGQGTLVTVSS.

The present invention successfully connects the Fc segment-containing fusion protein directly to the cell surface. The resulting effector cells have precise targeting properties. When expressing the Fc receptor, the effector cells to which the Fc segment-containing fusion protein is connected can be activated by the corresponding target cells. It has been demonstrated in the experiments that the NK cell expressing FcγRIII (CD16A) (NK92-FcγRIII) modified by the Fc segment-containing protein molecule can specifically kill target cells. Therefore, preparation of the effector cell with precise targeting by connecting the Fc segment-containing protein molecule directly to the surface of effector cells can, on the one hand, be simpler than the existing methods of preparing specific targeting cells by cell transfection, while reduce the risks that possibly arise from the genome manipulation of the effector cell; on the other hand, upon binding to antigens, the Fc segment of the fusion protein can activate the killing function of the effector cells by interacting with the Fc receptor. Therefore, the effector cells prepared based on the present invention have the advantages of simple preparation method, good safety and enhanced activity, and can be used to prevent and/or treat a disease caused by abnormal cell proliferation and/or function in the body, such as a tumor, an autoimmune disease and an infectious disease, and have important application values.

### DESCRIPTION OF THE DRAWINGS

FIG. 1: Schematic diagram of the structure of an Fc segment-containing fusion protein (including a recombinant antibody and an Fc fusion protein, No.: RP1-RP14).

FIG. 2: Polyacrylamide gel electrophoretogram of the recombinant antibody and Fc fusion protein solution.

FIG. 3: Graph of binding antigen activity assay of the recombinant antibody and Fc fusion protein.

FIG. 4: Flow cytometry profile showing the connection of the recombinant antibody or Fc fusion protein to NK92-FcγRIII cells using Sortase A.

FIG. 5: Flow cytometry profile showing the specific binding of cells connected with the recombinant antibody and Fc fusion protein to the corresponding target antigen.

FIG. 6: Flow cytometry profile showing that NK92-FcγRIII cells successfully connected to the recombinant antibody and Fc fusion protein can be further activated by target cells expressing specific antigens.

FIG. 7: Graph of cell killing effect, showing that NK92-FcγRIII cells connected to the recombinant antibody and Fc fusion protein can kill target cells expressing specific antigens.

FIG. 8: Flow cytometry profile showing the connection of the recombinant protein or Fc fusion protein to the surface of peripheral blood NK cells, peripheral blood T cells and cord blood NK cells using Sortase A.

### DETAILED DESCRIPTION

The present invention is based on the inventor's findings that the directly fusion and expression of the LPXTG region specifically recognized by Sortase A at the C-terminus of the intact antibody or Fc fusion protein using the protein modification method described in the existing patents and literatures (US20160122707A1; Jeong H J, et al., Generation of Ca2+-independent sortase A mutants with enhanced activity for protein and cell surface labeling. PLoS One. 2017 Dec. 4; 12(12):e0189068; Chen I, et al., A general strategy for the evolution of bond-forming enzymes using yeast display. Proc Natl Acad Sci USA. 2011 Jul. 12; 108(28):11399-404), i.e., no linking sequence added between the intact antibody or Fc fusion protein and the LPXTG sequence, is unable to achieve the direct connection of the intact antibody or Fc fusion protein to the cell surface by Sortase A. In contrast, using the fusion proteins in a specific configuration of the present invention, the intact antibody or Fc fusion protein may be directly connected to the cell surface via Sortase A.

As used herein, Sortase A is a membrane bound enzyme that enables a protein containing an enzyme substrate recognition sequence covalently linked to a bacterial cell membrane. The specific substrate recognition motif of Sortase A is LPXTG, and the enzyme cleaves between residues threonine (T) and glycine (G) of this substrate sequence, and further reacts with a substrate containing an oligoglycine sequence at the N-terminus for ligation. The type and source of Sortase A herein may not be limited as long as the Sortase A retains its functional properties as described above. For example, Sortase A may be a natural Sortase A or may be a variant of Sortase A (see, for example, CN201610726374.0).

As used herein, “first moiety” refers to the binding region in the fusion protein. The binding region may be an antigen-binding region of an antibody, a receptor for a ligand, or a ligand for a receptor, provided it is capable of binding a target, for example, on a target cell. The first moiety and the Fc region herein may be homologous or heterologous. For example, the first moiety and the Fc region are naturally occurring antibodies. The first moiety may be an antibody fragment. The first moiety may bind cancer antigens, infectious disease antigens, autoimmune disease antigens. For example, the first moiety may bind HER2 protein.

“Antibodies” which encompass a variety of antibody structures including, but not limited to, monoclonal antibodies, polyclonal antibodies, multispecific antibodies (e.g., bispecific antibodies), and antibody fragments, provided they display desired antigen-binding activities. Antibodies, also known as immunoglobulins (Igs for short), can be classified into five classes, IgG, IgM, IgA, IgE, and IgD, according to their physicochemical properties and biological functions. The Fab (fragment of antigen binding, Fab) segment of an antibody is an antigen-binding fragment, composed of a complete light chain and VH and CH1 domains of the heavy chain; the Fc segment is a crystallizable segment (fragment crystallizable, Fc), which region is composed of two or three constant domains of the heavy chains depending on the type of the antibody. For example, the Fc domain of IgG contains the heavy chain CH2 and CH3 domains.

The term “Fc segment” is used to define the C-terminal region of the immunoglobulin heavy chain, which contains at least a portion of the constant region. The term includes both a natural sequence Fc segment and a variant Fc segment. A “variant Fc segment” comprises an amino acid sequence that differs from a “natural” or “wild-type” sequence Fc segment due to at least one “amino acid modification”. The variant Fc region may have at least one amino acid substitution, for example, from about one to about ten amino acid substitutions, and preferably from about one to about five amino acid substitutions in the natural sequence Fc region or in the Fc segment of the parental polypeptide, as compared to the natural sequence Fc region or to the Fc segment of the parental polypeptide. The variant Fc segment herein will preferably have at least about 80% homology, and most preferably have at least about 90% homology, and more preferably have at least about 95% homology with the natural sequence Fc region and/or with the Fc segment of the parental polypeptide. The Fc region can bind various Fc receptors (FcRs) and other immune molecules, a process that triggers different target cell killing effects, including antibody-dependent cell-mediated cytotoxicity (ADCC), antibody-dependent cellular phagocytosis (ADCP), and complement-dependent cytotoxicity (CDC), and is the key effector segment of therapeutic antibody to exert its utility in vivo. The effector function of an antibody is a function contributed by the Fc effector domain of IgG (e.g., the Fc region of immunoglobulins). This function can be achieved, for example, by binding of the Fc effector domain to Fc receptors on immune cells with phagocytic or cytolytic activity, or by binding of the Fc effector domain to components of the complement system. Typical effector functions are ADCC, ADCP, and CDC. The “effector function” refers to those biological activities attributable to the Fc region of an antibody, which vary by antibody isotype. Examples of antibody effector functions include: C1q binding and complement-dependent cytotoxicity (CDC); Fc receptor binding; antibody-dependent cell-mediated cytotoxicity (ADCC); phagocytosis (ADCP); downregulation of cell surface receptors (e.g., B-cell receptors); and B-cell activation.

As used herein, the “linking moiety” refers to the moiety connecting the Fc region to the Sortase A substrate LPXTG. This moiety may comprise only a linker. In this case, the linking moiety may be the linker itself. Alternatively, the linking moiety may comprise only a protein or polypeptide, such as IL2 or scFv. The linking moiety may also comprise both the protein or polypeptide and the linker. For example, the linking moiety comprises IL2 or scFv and the linker, from the N-terminus to the C-terminus. There is no special restriction on the type of the linker provided that Fc-containing fusion proteins can be connected to the cell surface. For example, the linker can be a flexible linking sequence, such as G(n)S(n), n≥1, preferably, the linking sequence is GGGGSGGGGSGGGGS. The linker may also be a rigid linker sequence, such as (EAAAK)n, n≥1, preferably, the linking sequence is EAAAK. In an embodiment where the linking moiety comprises a protein or polypeptide and a linker, the linker is (GGGGS)n, n≥1, and the linker may also be a rigid linker sequence (EAAAK)n, n≥1. In an embodiment where the linking moiety comprises only a linker, the linker is (GGGGS)n, n≥3. In one embodiment, the linker is (EAAAK)ₙ, n≥1. Herein, n≥3 refers to, for example, an integer where n is 3, 4, 5, 6, 7, 8, 9, 10, or 11. n≥1 refers to, for example, an integer where n is 1, 2, 3, 4, 5, 6, 7, 8, 9, 10, or 11.

The term “moiety” is equivalent to “region” in the context used in a first moiety, a linking moiety, and a substrate moiety of Sortase A. The first moiety, the linking moiety and the substrate moiety may be referred to as the first region, the linking region and the substrate region.

The term “Fc receptor” or “FcR” is used to describe a receptor that binds to the Fc region of an antibody. The preferred FcR is a natural sequence human FcR. In addition, FcRs can be FcRs (gamma receptor) that bind IgG antibodies and include receptors for the subclasses FcγRI, FcγRII and FcγRIII, including allelic variants and alternative splice forms of these receptors. FcγRII receptors include FcγRIIA (activating receptor) and FcγRIIB (“inhibitory receptor”), which share similar amino acid sequences and differ mainly in their cytoplasmic domains. The activating receptor FcγRIIA comprises an immunoreceptor tyrosine activation motif (ITAM) in its cytoplasmic domain. The inhibitory receptor FcγRIIB comprises an immunoreceptor tyrosine inhibitory motif (ITIM) in its cytoplasmic domain. The “FcRs” encompasses other FcRs, including those to be identified in the future. The term also includes the neonatal receptor FcRn responsible for the transfer of maternal IgG to the fetus. Herein, the effector cells can express FcγRIII.

“Antibody-dependent cell-mediated cytotoxicity” and “ADCC” refer to cell-mediated responses in which non-specific cytotoxic cells (e.g., natural killer (NK) cells, neutrophils, and macrophages) expressing FcRs recognize antibodies bound on target cells and subsequently cause lysis of the target cells. The major cells (NK cells) used to mediate ADCC express only FcγRIII, while monocytes express FcγRI, FcγRII, and FcγRIII. FcR expression on hematopoietic cells is summarized in Table 3 on page 464 of Ravetch and Kinet, Annu. Rev. Immunol 9(1991)457-492.

The terms “antibody-dependent cellular phagocytosis” and “ADCP” refer to a process that antibody-coated cells are fully or partially internalized by phagocytic immune cells (e.g., macrophages, neutrophils, and dendritic cells) bound to the Fc region of immunoglobulins.

As used herein, “Fc fusion protein” or “fusion protein” refers to the fusion of the Fc region of an antibody with a protein molecule having a specific biological function using genetic engineering techniques, which not only has the original activity of the functional protein, but also has certain properties of the antibody, such as ADCC, CDC and ADCP. It can thus be seen that if an antigen-binding molecules with an Fc segment (such as an antibody and antigen-binding region-Fc fusion protein) is directly connected to the surface of an effector cell, not only can the cell have targeting for recognition of specific antigens, but also, the physiological effects triggered by the Fc region can further enhance the killing activity of the effector cell. Herein, the Fc fusion protein can have structuress of, from the N-terminus to the C-terminus:
full-length antibody-GGGGS-GGGGS-GGGGS-LPETGG;
full-length antibody-EAAAK-LPETGG;
full-length antibody-IL2-LPETGG;
full-length antibody-scFv-GGGGS-LPETGG;
full-length antibody-scFv-EAAAK-LPETGG;
scFv-Fc segment-GGGGS-GGGGS-GGGGS-LPETGG;
scFv-Fc segment-EAAAK-LPETGG.

The full-length antibody in these Fc fusion proteins can be IgG1, IgG2, IgG3 or IgG4 antibody.

“Antibody fragment” refers to a molecule of a non-intact antibody that comprises a portion of an intact antibody that binds the antigen to which the intact antibody binds. Examples of antibody fragments include, but are not limited to, Fv, Fab, Fab′, Fab′-SH, F(ab′)2; diabody; linear antibody; single chain antibody molecule (e.g., scFv); and multispecific antibody formed from an antibody fragment. Herein, the antibody fragment that binds a specific antigen may be used as the first moiety of the present invention.

### EXAMPLES

The present invention is described in further detail below in combination with specific examples, which are given merely to illustrate the invention and not to limit the scope of the invention.

The experimental methods in the following examples, if not otherwise specified, are conventional methods.

The materials, reagents and the like used in the following examples, if not otherwise specified, are available from commercial sources.

### Example 1: Expression and Purification of Sortase a Proteins

I. Construction of Recombinant Expression Plasmid pET-SrtA

1. The sequence of Sortase A gene is shown in Sequence 1, and primer F: 5′-CGGCAGCCATATGGCTAAACCTCAAATTCCGA-3′ (the underlined is the restriction recognition sequence of restriction endonuclease NdeI, sequence 35) and primer R: 5′-GTGGTGCTCGAGTTATTTGACTTCTGTAGCTAC-3′ (the underlined is the restriction recognition sequence of restriction endonuclease XhoI, sequence 36) were designed and synthesized according to Sequence 1.

Sequence 1:
(SEQ ID No. 1)
ATGGCTAAACCTCAAATTCCGAAAGATAAATCGAAAGTGGCAGGCTATAT
TGAAATTCCAGATGCTGATATTAAAGAACCAGTATATCCAGGACCAGCAA
CAAGCGAACAATTAAATAGAGGTGTAAGCTTTGCAGAAGAAAATGAATCA
CTAGATGATCAAAATATTTCAATTGCAGGACACACTTTCATTGACCGTCC
GAACTATCAATTTACAAATCTTAAAGCAGCCAAAAAAGGTAGTATGGTGT
ACTTTAAAGTTGGTAATGAAACACGTAAGTATAAAATGACAAGTATAAGA
AACGTTAAGCCTACAGATGTAGGAGTTCTAGATGAACAAAAAGGTAAAGA
TAAACAATTAACATTAATTACTTGTGATGATTACAATGAAAAGACAGGCG
TTTGGGAAACCCGTAAAATCTTTGTAGCTACAGAAGTCAAATAA

2. The DNA fragment shown in Sequence 1 above was synthesized and used as a template (reference: Chen I, et al., A general strategy for the evolution of bond-forming enzymes using yeast display. Proc Natl Acad Sci USA. 2011 Jul. 12; 108(28):11399-404.), and PCR amplification was performed using primer F and primer R synthesized in step 1 (reaction procedure: 95° C. for 5 min; 95° C. for 30 s, 55° C. for 30 s, 72° C. for 2 min, 30 cycles), and then the PCR amplification product of about 457 bp was recovered using a PCR product recovery kit.

3. The PCR amplification product recovered in step 2 was taken and digested with restriction endonuclease NdeI and restriction endonuclease XhoI, and the digested DNA fragment was recovered.

4. The vector pET-28(a) was digested with restriction endonuclease NdeI and restriction endonuclease XhoI, and the digested vector backbone was recovered.

5. The DNA fragment and the vector backbone were ligated to obtain the recombinant expression plasmid pET-SrtA.

The recombinant expression plasmid pET-SrtA was structurally described as follows: the small fragment between the restriction endonuclease NdeI and XhoI recognition sequences of vector pET-28(a) was replaced with the DNA molecule shown in Sequence 1 in the sequence listing. According to the sequencing results, the sequence SEQ ID No. 1 of Sortase A was correctly inserted into vector pET-28(a). The recombinant expression plasmid pET-SrtA expresses the protein shown in Sequence 2 in the sequence listing (hereinafter referred to as Sortase A protein).

Sequence 2:
(SEQ ID No. 2)
MGSSHHHHHHSSGLVPRGSHMAKPQIPKDKSKVAGYIEIPDADIKEPVYP
GPATSEQLNRGVSFAEENESLDDQNISIAGHTFIDRPNYQFTNLKAAKKG
SMVYFKVGNETRKYKMTSIRNVKPTDVGVLDEQKGKDKQLTLITCDDYNE
KTGVWETRKIFVATEVK

II. Expression and Purification of Sortase a Protein

1. The recombinant expression plasmid pET-SrtA was introduced into E. coli BL21(DE3) to obtain the recombinant strain, named BL21(DE3)-pET-SrtA.

2. The single colony of BL21(DE3)-pET-SrtA was inoculated into LB medium containing 100 mg/mL kanamycin, cultured at 37° C., 200 rpm with shaking, to obtain culture broth 1 with OD600 of 0.6. IPTG was added into culture broth 1 to obtain culture broth 2 (the concentration of IPTG is 0.1 mM in culture broth 2); then cultured at 37° C., 200 rpm with shaking for 6 h to obtain culture broth 3.

3. The culture broth 3 was taken for centrifugation at 5000 rpm for 10 min to collect the bacteria.

4. The bacteria collected in step 3 were taken and resuspended with 10 mL TBS buffer (10 mmol/L, Tris, 0.9% NaCl), pH7.4 to obtain bacterial suspension; then the bacterial suspension was ultrasonicated. Ultrasonication parameters: ultrasonication frequency 30%; ultrasonication for 10 s, stop for 5 s, total ultrasonication time is 30 min.

5. The system completed in step 4 was taken and centrifuged at 12000 rpm for 10 min to collect the supernatant.

6. The supernatant collected in step 5 was mixed with Ni-NTA Resin and incubated for 10 min, then the supernatant was dicarded, and the Ni-NTA Resin was washed with TBS buffer containing 20 mM imidazole, pH 7.4 for 3 times.

7. After step 6 was completed, elution was performed with TBS buffer containing 500 mM imidazole, pH 7.4 and the solution flowed through the column was collected, that is the Sortase A protein.

### Example 2: Preparation and Binding Activity Identification of Fc Segment-Containing Fusion Proteins

I. Preparation of Fc Segment-Containing Fusion Proteins

1. Construction of the Plasmid for Fc Segment-Containing Fusion Proteins

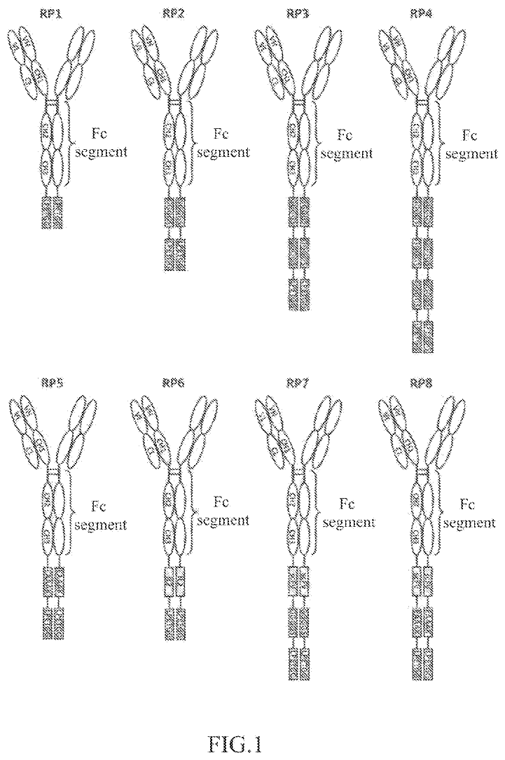

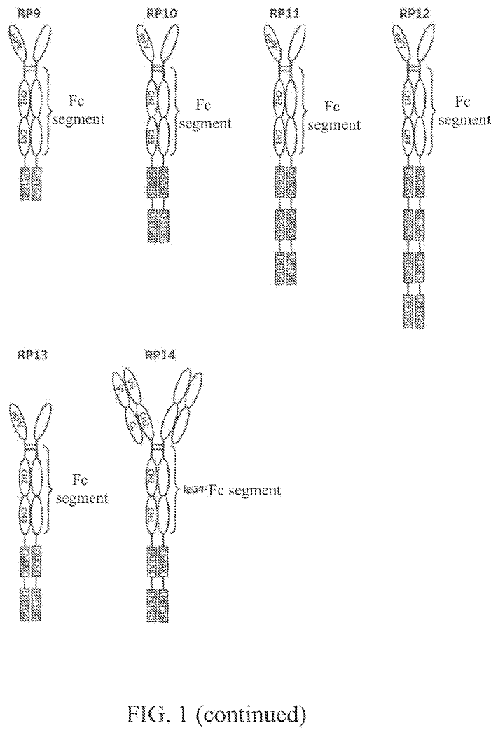

The structures of the Fc segment-containing fusion proteins (including recombinant antibodies and Fc fusion proteins, No. RP1-RP14) are shown in FIG. 1. The expression plasmids for the fusion proteins were constructed as described in Example 1. That is, the synthesized nucleic acids were cloned with primers containing HindIII and XhoI restriction sites and the synthesized sequences were ligated to the vector pCDNA3.1(+) digested with the corresponding enzymes, by the cloning technique as described in Example 1.

1.1. Construction of the Expression Plasmid for the Recombinant Antibody Ab-CH-LPETGG (No.: RP1):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab-CH-LPETGG (Sequence 4).

Sequence 3:
ATGGAGACCGACACCCTGCTGCTCTGGGTGCTGCTGCTCTGGGTGCCCGG
GTCGACCGGTGATATTCAGATGACCCAGAGCCCGAGCAGCCTGAGCGCGA
GCGTGGGCGATCGCGTGACCATTACCTGCCGCGCGAGCCAGGATGTGAAC
ACCGCGGTGGCGTGGTATCAGCAGAAACCGGGCAAAGCGCCGAAACTGCT
GATTTATAGCGCGAGCTTTCTGTATAGCGGCGTGCCGAGCCGCTTTAGCG
GCAGCCGCAGCGGCACCGATTTTACCCTGACCATTAGCAGCCTGCAGCCG
GAAGATTTTGCGACCTATTATTGCCAGCAGCATTATACCACCCCGCCGAC
CTTTGGCCAGGGCACCAAACTCGAGATCAAACGTACGGTGGCGGCGCCAT
CTGTCTTCATCTTCCCGCCATCTGATGAGCAGTTGAAATCTGGTACCGCT
AGCGTTGTGTGCCTGCTGAATAACTTCTATCCCAGAGAGGCCAAAGTACA
GTGGAAGGTGGATAACGCCCTCCAATCGGGTAACTCCCAGGAGAGTGTCA
CAGAGCAGGACAGCAAGGACAGCACCTACAGCCTCAGCAGCACCCTGACG
CTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGT
GTTGA (nucleotides 1-60 encode the signal peptide
and the last three nucleotides TGA are the stop
codon)
Sequence 4:
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The heavy chain expression vector was obtained by replacing the fragment between the HindIII and XhoI restriction sites in vector pCDNA3.1(+) with the DNA molecule shown in Sequence 5. In Sequence 5, nucleotides 61-1428 encode the full-length heavy chain of the recombinant antibody Ab-CH-LPETGG (Sequence 6).

Sequence 5:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAACTGCCCGAGACCGGCGGCTGA

(nucleotides 1-60 encode the signal peptide and the last three nucleotides TGA are the stop codon)

Sequence 6:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
LPETGG

1.2. Construction of the Expression Plasmid for the Recombinant Antibody Ab-CH-GGGGS-LPETGG (No.: RP2):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab-CH-GGGGS-LPETGG (Sequence 4).

The heavy chain expression vector was obtained by replacing the fragment between the HindIII and XhoI restriction sites in vector pCDNA3.1(+) with the DNA molecule shown in Sequence 7. In Sequence 7, nucleotides 61-1443 encode the full-length heavy chain of the recombinant antibody Ab-CH-GGGGS-LPETGG (Sequence 8).

Sequence 7:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAAGGCGGCGGCGGCAGCCTGCCCGAGACCGGCGGCTGA

(nucleotides 1-60 encode the signal peptide and the last three nucleotides TGA are the stop codon)

Sequence 8:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
GGGGSLPETGG

1.3. Construction of the Expression Plasmid for the Recombinant Antibody Ab-CH-GGGGS(x2)-LPETGG (No.: RP3):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab-CH-GGGGS(x2)-LPETGG (Sequence 4).

The heavy chain expression vector was obtained by replacing the fragment between the HindIII and XhoI restriction sites in vector pCDNA3.1(+) with the DNA molecule shown in Sequence 9. In Sequence 9, nucleotides 61-1473 encode the full-length heavy chain of the recombinant antibody Ab-CH-GGGGS(x2)-LPETGG (Sequence 10).

Sequence 9:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAAGGCGGCGGCGGCAGCGGCGGCGGCGGCAGCCTGCCCGAGA
CCGGCGGCTGA

(nucleotides 1-60 encode the signal peptide and the last three nucleotides TGA are the stop codon)

Sequence 10:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
GGGGSGGGGSLPETGG

1.4. Construction of the Expression Plasmid for the Recombinant Antibody Ab-CH-GGGGS(x3)-LPETGG (No.: RP4):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab-CH-GGGGS(x3)-LPETGG (Sequence 4).

The heavy chain expression vector was obtained by replacing the fragment between the HindIII and XhoI restriction sites in vector pCDNA3.1(+) with the DNA molecule shown in Sequence 11. In Sequence 11, nucleotides 61-1458 encode the full-length heavy chain of the recombinant antibody Ab-CH-GGGGS(x3)-LPETGG (Sequence 12).

Sequence 11:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAAGGCGGCGGCGGCAGCGGCGGCGGCGGCAGCGGCGGCGGCG
GCAGCCTGCCCGAGACCGGCGGCTGA

(nucleotides 1-60 encode the signal peptide and the last three nucleotides TGA are the stop codon)

Sequence 12:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
GGGGSGGGGSGGGGSLPETGG

1.5. Construction of the Expression Plasmid for the Recombinant Antibody Ab-CH-EAAAK-LPETGG (No.: RP5):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab-CH-EAAAK-LPETGG (Sequence 4).

The heavy chain expression vector was obtained by replacing the fragment between the HindIII and XhoI restriction sites in vector pCDNA3.1(+) with the DNA molecule shown in Sequence 13. In Sequence 13, nucleotides 61-1443 encode the full-length heavy chain of the recombinant antibody Ab-CH-EAAAK-LPETGG (Sequence 14).

Sequence 13:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAAGAGGCCGCCGCCAAGCTGCCCGAGACCGGCGGCTGA
Sequence 14:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
EAAAKLPETGG

1.6. Construction of the Expression Plasmid for the Recombinant Antibody Ab-CH-IL2-LPETGG (No. RP6):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab-CH-IL2-LPETGG (Sequence 4).

The recombinant heavy chain expression vector was obtained by replacing the fragment between the HindIII and XhoI restriction sites in vector pCDNA3.1(+) with the DNA molecule shown in Sequence 15. In Sequence 15, nucleotides 61-1854 encode the full-length heavy chain of the recombinant antibody Ab-CH-IL2-LPETGG (Sequence 16).

Sequence 15:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAAGGCGGCGGCGGCAGCGGATCCGCCCCCACCTCCTCCTCCA
CCAAGAAGACCCAGCTGCAGCTGGAGCACCTGCTGCTGGACCTGCAGATG
ATCCTGAACGGCATCAACAACTACAAGAACCCCAAGCTGACCAGGATGCT
GACCTTCAAGTTCTACATGCCCAAGAAGGCCACCGAGCTGAAGCACCTGC
AGTGCCTGGAGGAGGAGCTGAAGCCCCTGGAGGAGGTGCTGAACCTGGCC
CAGTCCAAGAACTTCCACCTGAGGCCCAGGGACCTGATCTCCAACATCAA
CGTGATCGTGCTGGAGCTGAAGGGCTCCGAGACCACCTTCATGTGCGAGT
ACGCCGACGAGACCGCCACCATCGTGGAGTTCCTGAACAGGTGGATCACC
TTCTGCCAGTCCATCATCTCCACCCTGACCCGTACGCTGCCCGAGACCGG
CGGCTGA
Sequence 16:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
GGGGSGSAPTSSSTKKTQLQLEHLLLDLQMILNGINNYKNPKLTRMLTFK
FYMPKKATELKHLQCLEEELKPLEEVLNLAQSKNFHLRPRDLISNINVIV
LELKGSETTFMCEYADETATIVEFLNRWITFCQSIISTLTRTLPETGG

(The underlined part is the amino acid sequence of IL2)

1.7. Construction of the Expression Plasmid for the Recombinant Antibody Ab1-CH-scFv.Ab2-GGGGS-LPETGG (No.: RP7):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab1-CH-scFv.Ab2-GGGGS-LPETGG (Sequence 4).

The recombinant heavy chain expression vector was obtained by replacing the fragment between the HindIII and XhoI restriction sites in vector pCDNA3.1(+) with the DNA molecule shown in Sequence 17. In Sequence 17, nucleotides 61-2178 encode the full-length heavy chain of the recombinant antibody Ab1-CH-scFv.Ab2-GGGGS-LPETGG (Sequence 18).

Sequence 17:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAAGGCGGCGGCGGCAGCGACATCCAGATGACCCAGAGCCCCA
GCAGCCTGAGCGCCAGCGTGGGCGACAGGGTGACCATCACCTGCAGGGCC
AGCCAGGACGTGAGCACCGCCGTGGCCTGGTACCAGCAGAAGCCCGGCAA
GGCCCCCAAGCTGCTGATCTACAGCGCCAGCTTCCTGTACAGCGGCGTGC
CCAGCAGGTTCAGCGGCAGCGGCAGCGGCACCGACTTCACCCTGACCATC
AGCAGCCTGCAGCCCGAGGACTTCGCCACCTACTACTGCCAGCAGTACCT
GTACCACCCCGCCACCTTCGGCCAGGGCACCAAGGTGGAGATCAAGGGCG
GCGGCGGCAGCGGCGGCGGCGGCAGCGGCGGCGGCGGCAGCGAGGTGCAG
CTGGTGGAGAGCGGCGGCGGCCTGGTGCAGCCCGGCGGCAGCCTGAGGCT
GAGCTGCGCCGCCAGCGGCTTCACCTTCAGCGACAGCTGGATCCACTGGG
TGAGGCAGGCCCCCGGCAAGGGCCTGGAGTGGGTGGCCTGGATCAGCCCC
TACGGCGGCAGCACCTACTACGCCGACAGCGTGAAGGGCAGGTTCACCAT
CAGCGCCGACACCAGCAAGAACACCGCCTACCTGCAGATGAACAGCCTGA
GGGCCGAGGACACCGCCGTGTACTACTGCGCCAGGAGGCACTGGCCCGGC
GGCTTCGACTACTGGGGCCAGGGCACCCTGGTGACCGTGAGCAGCGGCGG
CGGCGGCAGCCTGCCCGAGACCGGCGGCTGA

(the coding sequence for scFv.Ab2 sequence is underlined)

Sequence 18:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
GGGGSDIQMTQSPSSLSASVGDRVTITCRASQDVSTAVAWYQQKPGKAPK
LLIYSASFLYSGVPSRFSGSGSGTDFTLTISSLQPEDFATYYCQQYLYHP
ATFGQGTKVEIKGGGGSGGGGSGGGGSEVQLVESGGGLVQPGGSLRLSCA
ASGFTFSDSWIHWVRQAPGKGLEWVAWISPYGGSTYYADSVKGRFTISAD
TSKNTAYLQMNSLRAEDTAVYYCARRHWPGGFDYWGQGTLVTVSSGGGGS
LPETGG

(the scFv.Ab2 sequence is bolded and underlined)

1.8. Construction of the Expression Plasmid for the Recombinant Antibody Ab1-CH-scFv.Ab2-EAAAK-LPETGG (No.: RP8):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab1-CH-scFv.Ab2-EAAAK-LPETGG (Sequence 4).

The recombinant heavy chain expression vector was obtained by replacing the fragment between the HindIII and XhoI restriction sites in vector pCDNA3.1(+) with the DNA molecule shown in Sequence 19. In Sequence 19, nucleotides 61-2178 encode the full-length heavy chain of the recombinant antibody Ab1-CH-scFv.Ab2-EAAAK-LPETGG (Sequence 20).

Sequence 19:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTGTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
CCCGGGTAAAGGCGGCGGCGGCAGCGACATCCAGATGACCCAGAGCCCCA
GCAGCCTGAGCGCCAGCGTGGGCGACAGGGTGACCATCACCTGCAGGGCC
AGCCAGGACGTGAGCACCGCCGTGGCCTGGTACCAGCAGAAGCCCGGCAA
GGCCCCCAAGCTGCTGATCTACAGCGCCAGCTTCCTGTACAGCGGCGTGC
CCAGCAGGTTCAGCGGCAGCGGCAGCGGCACCGACTTCACCCTGACCATC
AGCAGCCTGCAGCCCGAGGACTTCGCCACCTACTACTGCCAGCAGTACCT
GTACCACCCCGCCACCTTCGGCCAGGGCACCAAGGTGGAGATCAAGGGCG
GCGGCGGCAGCGGCGGCGGCGGCAGCGGCGGCGGCGGCAGCGAGGTGCAG
CTGGTGGAGAGCGGCGGCGGCCTGGTGCAGCCCGGCGGCAGCCTGAGGCT
GAGCTGCGCCGCCAGCGGCTTCACCTTCAGCGACAGCTGGATCCACTGGG
TGAGGCAGGCCCCCGGCAAGGGCCTGGAGTGGGTGGCCTGGATCAGCCCC
TACGGCGGCAGCACCTACTACGCCGACAGCGTGAAGGGCAGGTTCACCAT
CAGCGCCGACACCAGCAAGAACACCGCCTACCTGCAGATGAACAGCCTGA
GGGCCGAGGACACCGCCGTGTACTACTGCGCCAGGAGGCACTGGCCCGGC
GGCTTCGACTACTGGGGCCAGGGCACCCTGGTGACCGTGAGCAGCGAGGC
CGCCGCCAAGCTGCCCGAGACCGGCGGCTGA

(the scFv.Ab2 sequence is underlined, and the linker sequence is GAGGCCGCCGCCAAG)

Sequence 20:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
GGGGSDIQMTQSPSSLSASVGDRVTITCRASQDVSTAVAWYQQKPGKAPK
LLIYSASFLYSGVPSRFSGSGSGTDFTLTISSLQPEDFATYYCQQYLYHP
ATFGQGTKVEIKGGGGSGGGGSGGGGSEVQLVESGGGLVQPGGSLRLSCA
ASGFTFSDSWIHWVRQAPGKGLEWVAWISPYGGSTYYADSVKGRFTISAD
TSKNTAYLQMNSLRAEDTAVYYCARRHWPGGFDYWGQGTLVTVSS [symbol]
LPETGG

(the scFv.Ab2 sequence is bold and underlined, and the linker sequence is EAAAK)

1.9. Construction of the Expression Plasmid for the Fc Fusion Protein scFv-Fc-LPETGG (No. RP9):

The Fc fusion protein expression vector was obtained by replacing the fragment between the HindIII and XhoI restriction sites in vector pCDNA3.1(+) with the DNA molecule shown in Sequence 21. In Sequence 21, nucleotides 61-1506 encode the full-length protein of scFv-Fc-LPETGG (Sequence 22).

Sequence 21:
ATGGAGACCGACACCCTGCTGCTCTGGGTGCTGCTGCTCTGGGTGCCCGG
GTCGACCGGTGATATTCAGATGACCCAGAGCCCGAGCAGCCTGAGCGCGA
GCGTGGGCGATCGCGTGACCATTACCTGCCGCGCGAGCCAGGATGTGAAC
ACCGCGGTGGCGTGGTATCAGCAGAAACCGGGCAAAGCGCCGAAACTGCT
GATTTATAGCGCGAGCTTTCTGTATAGCGGCGTGCCGAGCCGCTTTAGCG
GCAGCCGCAGCGGCACCGATTTTACCCTGACCATTAGCAGCCTGCAGCCG
GAAGATTTTGCGACCTATTATTGCCAGCAGCATTATACCACCCCGCCGAC
CTTTGGCCAGGGCACCAAACTCGAGATCAAAGGCGGCGGCGGCAGCGGCG
GCGGCGGCAGCGGCGGCGGCGGCAGCGAAGTGCAGCTGGTGGAAAGCGGC
GGCGGCCTGGTGCAGCCGGGCGGCAGCCTGCGCCTGAGCTGCGCGGCGAG
CGGCTTTAACATTAAAGATACCTATATTCATTGGGTGCGCCAGGCGCCGG
GCAAAGGCCTGGAATGGGTGGCGCGCATTTATCCGACCAACGGCTATACC
CGCTATGCGGATAGCGTGAAAGGCCGCTTTACCATTAGCGCGGATACCAG
CAAAAACACCGCGTATCTGCAGATGAACAGCCTGCGCGCGGAAGATACCG
CGGTGTATTATTGCAGCCGCTGGGGCGGCGATGGCTTTTATGCGATGGAT
TATTGGGGCCAGGGCACCCTGGTGACCGTGAGCAGCGCTAGCGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCCCCGGGTAAACTGCCCGAGACC
GGCGGCTGA
Sequence 22:
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKLEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSASEPKSCD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHEALHNHYTQKSLSLSPGKLPETGG

1.10. Construction of the Expression Plasmid for the Fc Fusion Protein scFv-Fc-GGGGS-LPETGG (No.: RP10):

The Fc fusion protein expression vector was obtained by replacing the fragment between the HindIII and XhoI restriction sites in vector pCDNA3.1(+) with the DNA molecule shown in Sequence 23. In Sequence 23, nucleotides 61-1521 encode the full-length protein of scFv-Fc-GGGGS-LPETGG (Sequence 24).

Sequence 23:
ATGGAGACCGACACCCTGCTGCTCTGGGTGCTGCTGCTCTGGGTGCCCGG
GTCGACCGGTGATATTCAGATGACCCAGAGCCCGAGCAGCCTGAGCGCGA
GCGTGGGCGATCGCGTGACCATTACCTGCCGCGCGAGCCAGGATGTGAAC
ACCGCGGTGGCGTGGTATCAGCAGAAACCGGGCAAAGCGCCGAAACTGCT
GATTTATAGCGCGAGCTTTCTGTATAGCGGCGTGCCGAGCCGCTTTAGCG
GCAGCCGCAGCGGCACCGATTTTACCCTGACCATTAGCAGCCTGCAGCCG
GAAGATTTTGCGACCTATTATTGCCAGCAGCATTATACCACCCCGCCGAC
CTTTGGCCAGGGCACCAAACTCGAGATCAAAGGCGGCGGCGGCAGCGGCG
GCGGCGGCAGCGGCGGCGGCGGCAGCGAAGTGCAGCTGGTGGAAAGCGGC
GGCGGCCTGGTGCAGCCGGGCGGCAGCCTGCGCCTGAGCTGCGCGGCGAG
CGGCTTTAACATTAAAGATACCTATATTCATTGGGTGCGCCAGGCGCCGG
GCAAAGGCCTGGAATGGGTGGCGCGCATTTATCCGACCAACGGCTATACC
CGCTATGCGGATAGCGTGAAAGGCCGCTTTACCATTAGCGCGGATACCAG
CAAAAACACCGCGTATCTGCAGATGAACAGCCTGCGCGCGGAAGATACCG
CGGTGTATTATTGCAGCCGCTGGGGCGGCGATGGCTTTTATGCGATGGAT
TATTGGGGCCAGGGCACCCTGGTGACCGTGAGCAGCGCTAGCGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCCCCGGGTAAAGGCGGCGGCGGC
AGCCTGCCCGAGACCGGCGGCTGA
Sequence 24:
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKLEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSASEPKSCD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHEALHNHYTQKSLSLSPGKGGGGSLPETGG

1.11. Construction of the Expression Plasmids for the Fc Fusion Protein scFv-Fc-(GGGGS)x2-LPETGG (No.: RP11):

The Fc fusion protein expression vector was obtained by replacing the fragment between the HindIII and XhoI restriction sites in vector pCDNA3.1(+) with the DNA molecule shown in Sequence 25. In Sequence 25, nucleotides 61-1536 encode the full-length protein of scFv-Fc-(GGGGS)x2-LPETGG (Sequence 26).

Sequence 25:
ATGGAGACCGACACCCTGCTGCTCTGGGTGCTGCTGCTCTGGGTGCCCGG
GTCGACCGGTGATATTCAGATGACCCAGAGCCCGAGCAGCCTGAGCGCGA
GCGTGGGCGATCGCGTGACCATTACCTGCCGCGCGAGCCAGGATGTGAAC
ACCGCGGTGGCGTGGTATCAGCAGAAACCGGGCAAAGCGCCGAAACTGCT
GATTTATAGCGCGAGCTTTCTGTATAGCGGCGTGCCGAGCCGCTTTAGCG
GCAGCCGCAGCGGCACCGATTTTACCCTGACCATTAGCAGCCTGCAGCCG
GAAGATTTTGCGACCTATTATTGCCAGCAGCATTATACCACCCCGCCGAC
CTTTGGCCAGGGCACCAAACTCGAGATCAAAGGCGGCGGCGGCAGCGGCG
GCGGCGGCAGCGGCGGCGGCGGCAGCGAAGTGCAGCTGGTGGAAAGCGGC
GGCGGCCTGGTGCAGCCGGGCGGCAGCCTGCGCCTGAGCTGCGCGGCGAG
CGGCTTTAACATTAAAGATACCTATATTCATTGGGTGCGCCAGGCGCCGG
GCAAAGGCCTGGAATGGGTGGCGCGCATTTATCCGACCAACGGCTATACC
CGCTATGCGGATAGCGTGAAAGGCCGCTTTACCATTAGCGCGGATACCAG
CAAAAACACCGCGTATCTGCAGATGAACAGCCTGCGCGCGGAAGATACCG
CGGTGTATTATTGCAGCCGCTGGGGCGGCGATGGCTTTTATGCGATGGAT
TATTGGGGCCAGGGCACCCTGGTGACCGTGAGCAGCGCTAGCGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCCCCGGGTAAAGGCGGCGGCGGC
AGCGGCGGCGGCGGCAGCCTGCCCGAGACCGGCGGCTGA
Sequence 26:
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKLEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSASEPKSCD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHEALHNHYTQKSLSLSPGKGGGGSGGGGSLPETGG

1.12. Construction of the Expression Plasmid for the Fc Fusion Protein scFv-Fc-(GGGGS)x3-LPETGG (No.: RP12):

The Fc fusion protein expression vector was obtained by replacing the fragment between the HindIII and XhoI restriction sites in vector pCDNA3.1(+) with the DNA molecule shown in Sequence 27. In Sequence 27, nucleotides 61-1550 encode the full-length protein of scFv-Fc-(GGGGS)x3-LPETGG (Sequence 28).

Sequence 27:
ATGGAGACCGACACCCTGCTGCTCTGGGTGCTGCTGCTCTGGGTGCCCGG
GTCGACCGGTGATATTCAGATGACCCAGAGCCCGAGCAGCCTGAGCGCGA
GCGTGGGCGATCGCGTGACCATTACCTGCCGCGCGAGCCAGGATGTGAAC
ACCGCGGTGGCGTGGTATCAGCAGAAACCGGGCAAAGCGCCGAAACTGCT
GATTTATAGCGCGAGCTTTCTGTATAGCGGCGTGCCGAGCCGCTTTAGCG
GCAGCCGCAGCGGCACCGATTTTACCCTGACCATTAGCAGCCTGCAGCCG
GAAGATTTTGCGACCTATTATTGCCAGCAGCATTATACCACCCCGCCGAC
CTTTGGCCAGGGCACCAAACTCGAGATCAAAGGCGGCGGCGGCAGCGGCG
GCGGCGGCAGCGGCGGCGGCGGCAGCGAAGTGCAGCTGGTGGAAAGCGGC
GGCGGCCTGGTGCAGCCGGGCGGCAGCCTGCGCCTGAGCTGCGCGGCGAG
CGGCTTTAACATTAAAGATACCTATATTCATTGGGTGCGCCAGGCGCCGG
GCAAAGGCCTGGAATGGGTGGCGCGCATTTATCCGACCAACGGCTATACC
CGCTATGCGGATAGCGTGAAAGGCCGCTTTACCATTAGCGCGGATACCAG
CAAAAACACCGCGTATCTGCAGATGAACAGCCTGCGCGCGGAAGATACCG
CGGTGTATTATTGCAGCCGCTGGGGCGGCGATGGCTTTTATGCGATGGAT
TATTGGGGCCAGGGCACCCTGGTGACCGTGAGCAGCGCTAGCGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCCCCGGGTAAAGGCGGCGGCGGC
AGCGGCGGCGGCGGCAGCGGCGGCGGCGGCAGCCTGCCCGAGACCGGCGG
CTGA
Sequence 28:
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKLEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSASEPKSCD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHEALHNHYTQKSLSLSPGKGGGGSGGGGSGGGGSLPETGG

1.13. Construction of the Expression Plasmid for the Fc Fusion Protein scFv-Fc-EAAAK-LPETGG (No.: RP13):

The Fc fusion protein expression vector was obtained by replacing the fragment between the HindIII and XhoI restriction sites in vector pCDNA3.1(+) with the DNA molecule shown in Sequence 29. In Sequence 29, nucleotides 61-1521 encode the full-length protein of scFv-Fc-EAAAK-LPETGG (Sequence 30).

Sequence 29:
ATGGAGACCGACACCCTGCTGCTCTGGGTGCTGCTGCTCTGGGTGCCCGG
GTCGACCGGTGATATTCAGATGACCCAGAGCCCGAGCAGCCTGAGCGCGA
GCGTGGGCGATCGCGTGACCATTACCTGCCGCGCGAGCCAGGATGTGAAC
ACCGCGGTGGCGTGGTATCAGCAGAAACCGGGCAAAGCGCCGAAACTGCT
GATTTATAGCGCGAGCTTTCTGTATAGCGGCGTGCCGAGCCGCTTTAGCG
GCAGCCGCAGCGGCACCGATTTTACCCTGACCATTAGCAGCCTGCAGCCG
GAAGATTTTGCGACCTATTATTGCCAGCAGCATTATACCACCCCGCCGAC
CTTTGGCCAGGGCACCAAACTCGAGATCAAAGGCGGCGGCGGCAGCGGCG
GCGGCGGCAGCGGCGGCGGCGGCAGCGAAGTGCAGCTGGTGGAAAGCGGC
GGCGGCCTGGTGCAGCCGGGCGGCAGCCTGCGCCTGAGCTGCGCGGCGAG
CGGCTTTAACATTAAAGATACCTATATTCATTGGGTGCGCCAGGCGCCGG
GCAAAGGCCTGGAATGGGTGGCGCGCATTTATCCGACCAACGGCTATACC
CGCTATGCGGATAGCGTGAAAGGCCGCTTTACCATTAGCGCGGATACCAG
CAAAAACACCGCGTATCTGCAGATGAACAGCCTGCGCGCGGAAGATACCG
CGGTGTATTATTGCAGCCGCTGGGGCGGCGATGGCTTTTATGCGATGGAT
TATTGGGGCCAGGGCACCCTGGTGACCGTGAGCAGCGCTAGCGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCCCCGGGTAAAGAGGCCGCCGCC
AAGCTGCCCGAGACCGGCGGCTGA
Sequence 30:
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKLEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSASEPKSCD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHEALHNHYTQKSLSLSPGKEAAAKLPETGG

1.14. Construction of the Expression Plasmid for the Recombinant Antibody Ab(IgG4)-EAAAK-LPETGG (No.: RP14):

The light chain expression vector was obtained by replacing the fragment between the HindIII and XhoI restriction sites in vector pCDNA3.1(+) with the DNA molecule shown in Sequence 3. In Sequence 3, nucleotides 61-702 encode the full-length light chain of the recombinant antibody Ab(IgG4)-EAAAK-LPETGG (Sequence 4).

The heavy chain expression vector was obtained by replacing the fragment between the HindIII and XhoI restriction sites in vector pCDNA3.1(+) with the DNA molecule shown in Sequence 31. In Sequence 31, nucleotides 61-1800 encode the full-length heavy chain of the recombinant antibody Ab(IgG4)-EAAAK-LPETGG (Sequence 32).

Sequence 31:
ATGGAGACCGACACCCTGCTGCTCTGGGTGCTGCTGCTCTGGGTGCCCGG
GTCGACCGGTGAAGTGCAGCTGGTGGAAAGCGGCGGCGGCCTGGTGCAGC
CGGGCGGCAGCCTGCGCCTGAGCTGCGCGGCGAGCGGCTTTAACATTAAA
GATACCTATATTCATTGGGTGCGCCAGGCGCCGGGCAAAGGCCTGGAATG
GGTGGCGCGCATTTATCCGACCAACGGCTATACCCGCTATGCGGATAGCG
TGAAAGGCCGCTTTACCATTAGCGCGGATACCAGCAAAAACACCGCGTAT
CTGCAGATGAACAGCCTGCGCGCGGAAGATACCGCGGTGTATTATTGCAG
CCGCTGGGGCGGCGATGGCTTTTATGCGATGGATTATTGGGGCCAGGGCA
CCCTGGTGACCGTGAGCAGCGCTAGCACCAAGGGCCCATCGGTCTTCCCC
CTGGCACCCTGCTCCCGCAGTACTTCTGAGAGCACAGCGGCCCTGGGCTG
CCTGGTCAAGGACTACTTCCCCGAACCGGTGACGGTGTCGTGGAACTCAG
GCGCCCTGACCAGCGGCGTGCACACCTTCCCGGCTGTCCTACAGTCCTCA
GGACTCTACTCCCTCAGCAGCGTGGTGACTGTGCCCTCTAGCAGCTTGGG
CACCAAGACCTACACGTGCAACGTGGATCACAAGCCCAGCAACACCAAGG
TGGACAAACGCGTTGAGTCCAAATATGGTCCCCCATGCCCACCATGCCCA
GCACCTGAGTTCCTGGGGGGACCATCAGTCTTCCTGTTCCCCCCAAAACC
CAAGGACACTCTCATGATCTCCCGGACCCCTGAGGTCACGTGCGTGGTGG
TGGACGTGAGCCAGGAAGACCCCGAGGTCCAGTTCAACTGGTACGTGGAT
GGCGTGGAGGTGCATAATGCCAAGACAAAGCCGCGGGAGGAGCAGTTCAA
CAGCACGTACCGTGTGGTCAGCGTCCTCACCGTCCTGCACCAGGACTGGC
TGAACGGCAAGGAGTACAAGTGCAAGGTCTCCAACAAAGGCCTCCCGTCC
TCCATCGAGAAAACCATCTCCAAAGCCAAAGGGCAGCCCCGAGAGCCACA
GGTGTACACCCTGCCCCCATCCCAGGAGGAGATGACCAAGAACCAGGTCA
GCCTGACCTGCCTGGTCAAAGGCTTCTACCCCAGCGACATCGCCGTGGAG
TGGGAGAGCAATGGGCAGCCGGAGAACAACTACAAGACCACGCCTCCCGT
GCTGGACTCCGACGGCTCCTTCTTCCTCTACAGCAGGCTAACCGTGGACA
AGAGCAGGTGGCAGGAGGGGAATGTCTTCTCATGCTCCGTGATGCATGAG
GCTCTGCACAACCACTACACACAGAAGAGCCTCTCCCTGTCTCTGGGTAA
AGAGGCCGCCGCCAAGCTGCCCGAGACCGGCGGCTGA
Sequence 32:
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKEAA
AKLPETGG

2. Preparation of Fc Segment-Containing Fusion Proteins

2.1 After equimolar mixing of the above heavy chain expression vectors and light chain expression vectors (if there is a light chain), CHO cells were cotransfected using ExpiFectamine™ CHO Transfection Kit (purchased from Therno Fisher, Inc., Cat. No.: A29129) (operated according to the instructions) to express recombinant antibodies; CHO cells were transfected with the above Fc fusion protein expression plasmids using ExpiFectamine™ CHO Transfection Kit (Cat. No.: A29129) (operated according to the instructions) to express Fc fusion proteins; CHO cells were then cultured at 37° C. and 5% CO₂ using ExpiCHO™ Expression Medium for 8 days, and the supernatant was collected.

2.2 The supernatant obtained in step 2.1 was taken and purified with Protein A. The purified product was collected.

2.3 The purified product obtained in step 2.2 was taken and subjected to ultra-filtration concentration and exchange. The system was replaced with PBS buffer to obtain the solution of recombinant antibodies and Fc fusion proteins.

The protein concentration of the solution was detected by UV absorption method at A280 nm.

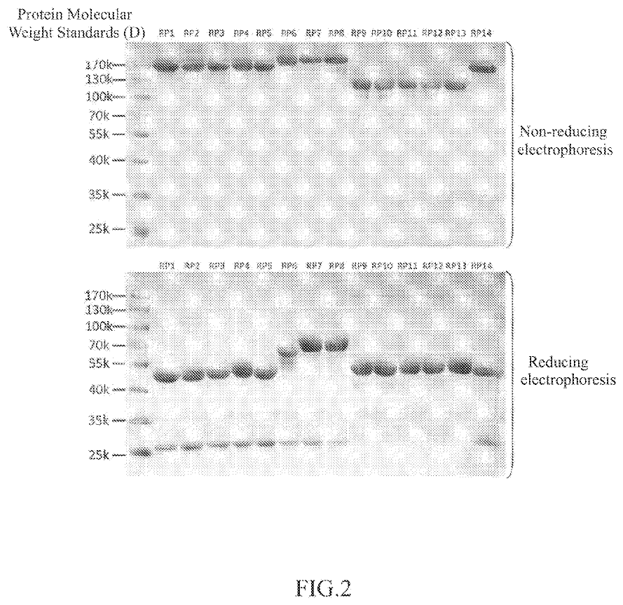

The polyacrylamide gel electrophoresis of the solution of Fc segment-containing fusion proteins (including recombinant antibodies and Fc fusion proteins) is shown in FIG. 2. In FIG. 2, the non-reducing electrophoresis is without the addition of reducing agent 2-mercaptoethanol, and the reducing electrophoresis is with the addition of 1% reducing agent 2-mercaptoethanol. As shown in FIG. 2, the apparent molecular weights of the recombinant antibodies and the Fc fusion proteins are consistent with their predicted molecular weights, and the Fc segment-containing fusion proteins were correctly expressed.

II. ELISA Assay for the Binding Activity of Fc Segment-Containing Fusion Proteins 1. A microtiter plate was taken and the coating solution (100 μl/well) was added overnight at 4° C.

The coating solution consists of a coating antigen and the coating buffer, and the concentration of the coating antigen in the coating solution is 1 μg/mL. The coating antigen is HER2 protein (purchased from Sino Biological Inc., Cat. No.: 10004-H08H4). The coating buffer (pH 9.6): Na₂CO₃ 1.59 g, NaHCO₃ 2.94 g, the balance being water.

2, After completing step 1, the microtiter plate was taken and washed with PBST buffer for three times.

3. After completing step 2, the microtiter plate was taken and added with PBST buffer containing 5 g/100 mL skimmed milk powder, and blocked at 37° C. for 1 h.

4. The Fc segment-containing fusion protein solution prepared above was taken to prepare the stock solution with antibody concentration of 10 μg/mL by using PBST buffer containing 5 g/100 mL skimmed milk powder, followed by three-fold gradient dilution using PBST buffer containing 5 g/100 mL skimmed milk powder to obtain Fc segment-containing fusion protein solutions at different concentrations.

5. The microtiter plate after completing step 3 was taken and added with Fc segment-containing fusion protein solutions (100 μL per well) at different concentrations obtained in step 4, and incubated at 37° C. for 1 h. 3 replicate wells were set for each concentration.

6. After completing step 5, the microtiter plate was taken and washed with PBST buffer for three times (250 μL per well each time).

7. After completing step 6, the microtiter plate was taken and added with HRP-labeled goat anti-human IgG secondary antibody dilution (HRP-labeled goat anti-human IgG secondary antibody diluted 1:40,000 in PBST buffer containing 5 g/100 mL skimmed milk powder), and incubated at 37° C. for 30 min.

8. After completing step 7, the microtiter plate was taken and added with TMB chromogenic reagent (100 μL per well), and developed at room temperature for 10 min.

9. After completing step 8, the microtiter plate was taken and the development was terminated by adding 2N H₂SO₄ solution (50 μL per well), and then the OD value was detected at a wavelength of 450 nm.

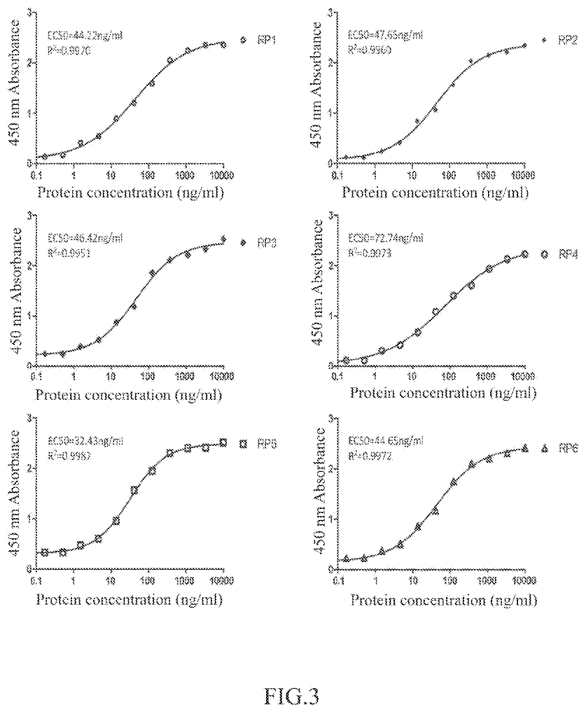

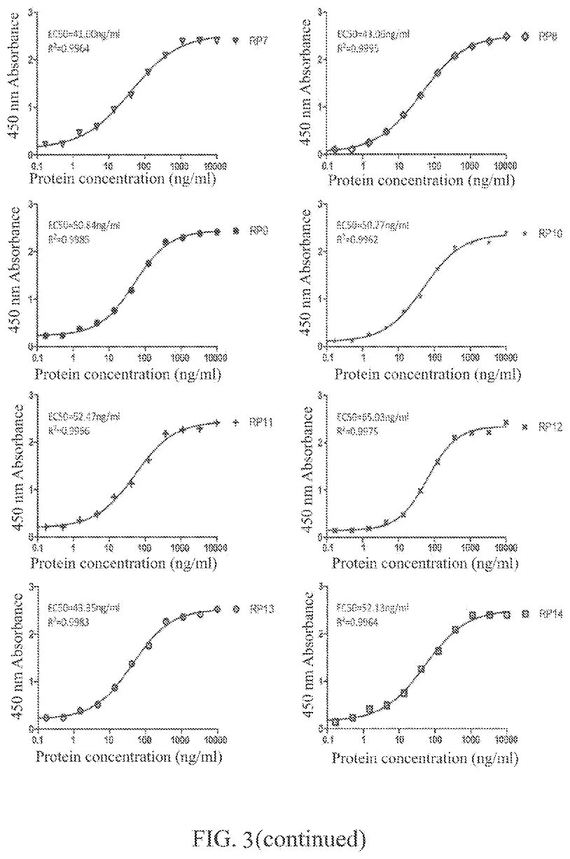

The results are shown in FIG. 3. The results show that the Fc segment-containing fusion proteins prepared above can bind to the corresponding antigen with high binding activity. This indicates that the above modification of the Fc segment-containing fusion proteins did not alter their antigen-binding activity.

### Example 3: Connection of the Fc Segment-Containing Fusion Proteins to NK92-FcγRIII Cells Using Sortase a and Cell Activity Assay

I. Connection of the Fc Segment-Containing Fusion Proteins to NK92-FcγRIII Cells (Purchased from ATCC, Cat. No.: pta-8837) Using Sortase A

1. 100 μL of NK92-FcγRIII cell suspension at a concentration of 1.0×10⁶/mL was taken and added with Sortase A to a final concentration of 20 ug/ml, and the Fc segment-containing fusion protein to a final concentration of 10 ug/ml, to obtain an incubation system. The incubation system was incubated at 25° C. for 90 min. The cells were collected after centrifugation and washed thoroughly with 0.01 mol/L PBS buffer, pH 7.4. The cells obtained above were named as: recombinant protein-SrtA-NK.

2. 100 μL of NK92-FcγRIII cell suspension at a concentration of 1.0×10⁶/mL was taken and added with the Fc segment-containing fusion protein alone to a final concentration of 10 ug/ml to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-NK.

3. The cells collected in steps 1 and 2 were taken and added with APC-labeled Donkey Anti-Human IgG antibody (APC AffiniPure F(ab′)₂ Fragment Donkey Anti-Human IgG, Fcγ fragment specific, purchased from Jackson ImmunoResearch Company, Cat. No.: 709-136-098) according to the instructions to obtain an incubation system. The incubation system was incubated at 25° C. for 30 min.

4. After completing step 3, cells were collected by centrifugation, and washed thoroughly with 0.01 mol/L PBS buffer, pH 7.4.

5. After completing step 4, cells without treatment were used as blank group cells, and the levels of cells labeled with Fc segment-containing fusion proteins with or without the addition of Sortase A were detected using flow cytometry.

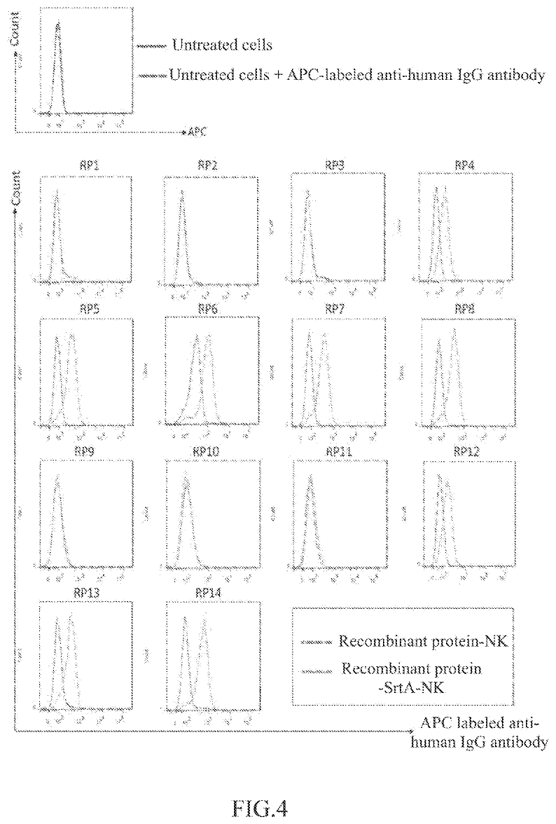

The experimental results are shown in FIG. 4. The results indicate that Sortase A cannot connect the Fc segment-containing fusion proteins with LPXTG sequence added only at the C-terminus of the Fc segment (e.g. recombinant antibody RP1 or Fc fusion protein RP9) to the cell surface; the recombinant antibodies (RP4, RP5, RP6, RP7, RP8, RP14) and Fc fusion proteins (RP12, RP13) modified by further adding a linking sequence between the Fc segment and LPXTG region, can be successfully connected to the cell surface mediated by Sortase A.

II. Cells Connected with Fc Segment-Containing Fusion Proteins can Bind Specifically to the Corresponding Target Antigens

1. 200 μL of NK92-FcγRIII cell suspension at a concentration of 1.0×10⁶/mL was taken and added with Sortase A to a final concentration of 20 ug/ml and Fc segment-containing fusion protein to a final concentration of 10 ug/ml, to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-SrtA-NK.

2. 200 μL of NK92-FcγRIII cell suspension at a concentration of 1.0×10⁶/mL was taken and added with the Fc segment-containing fusion protein alone to a final concentration of 10 ug/ml to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-NK.

3. 100 μL of the cell suspension after the completion of steps 1 and 2 at a concentration of 1.0×10⁶/mL was taken and added with biotin-labeled HER2 protein (purchased from ACRObiosysterms, Cat. No.: H822R) to obtain an incubation system. The incubation system was incubated at 25° C. for 60 min.

4. After completing step 3, cells were collected by centrifugation, and washed thoroughly with 0.01 mol/L PBS buffer, pH 7.4.

5. After completing step 4, the incubation system was obtained by adding FITC-labeled streptavidin (FITC Streptavidin, purchased from Biolegend Company, Cat. No.: 405201) according to the instructions. The incubation system was incubated at 25° C. for 30 min.

6. After completing step 5, cells were collected by centrifugation, and washed thoroughly with 0.01 mol/L PBS buffer, pH 7.4.

7. After completing step 6, cells without treatment were used as blank group cells, and the levels of binding of cells to target antigen HER2 were detected using flow cytometry.

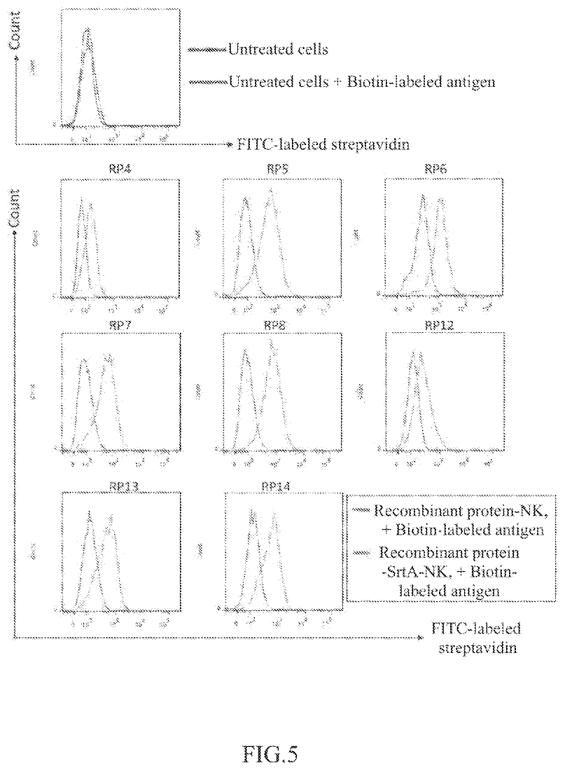

The experimental results are shown in FIG. 5. The results indicate that the cells successfully connected with the Fc segment-containing fusion proteins can bind specifically to the target antigens.

III. NK92-FcγRIII Cells Successfully Connected with the Fc Segment-Containing Fusion Proteins can be Further Activated by Target Cells Expressing Specific Antigens.

1. 400 μL of NK92-FcγRIII cell suspension at a concentration of 1.0×10⁶/mL was taken and added with Sortase A to a final concentration of 20 ug/ml and Fc segment-containing fusion proteins to a final concentration of 10 ug/ml, to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-SrtA-NK.

2. 200 μL of NK92-FcγRIII cell suspension at a concentration of 1.0×10⁶/mL was taken and added with the Fc segment-containing fusion proteins alone to a final concentration of 10 ug/ml to obtain an incubation system. The incubation system was incubated for 90 min at 25° C. The cells were collected by centrifugation and washed thoroughly with 0.01 mol/L PBS buffer, pH 7.4. The cells obtained above were named as: recombinant protein-NK.

3. A 24-well plate was taken and added with 200 μL of suspension of SKOV3 cells (which is an antigen HER2 protein high-expressing cell line) at a concentration of 4.0×10⁵/mL, and then with 200 μL of cell suspension after the completion of step 1 or 2 at a concentration of 2×10⁶/mL to obtain an incubation system. The incubation system was incubated at 37° C. for 60 min.

4. The system after completing step 3 was taken, and cells were collected by centrifugation at 4° C. and washed thoroughly with pH 7.4, 0.01 mol/L PBS buffer in ice bath.

5. After completing step 4, Alexa Fluor® 488-labeled anti-human CD107a antibody (Alexa Fluor® 488anti-human CD107a, purchased from Biolegend Company, Cat. No.: 328610) and APC-labeled anti-human CD56 antibody (APC anti-human CD56 (NCAM), purchased from Biolegend Company, Cat. No.: 318310) were added according to the instructions to obtain an incubation system. The incubation system was incubated at 4° C. for 30 min.

6. After completing step 5, cells were collected by centrifugation at 4° C., and wash thoroughly with pH 7.4, 0.01 mol/L PBS buffer in an ice bath.

7. After completing step 6, cells without treatment were used as blank group cells, and the level of cellular CD107a was detected using flow cytometry.

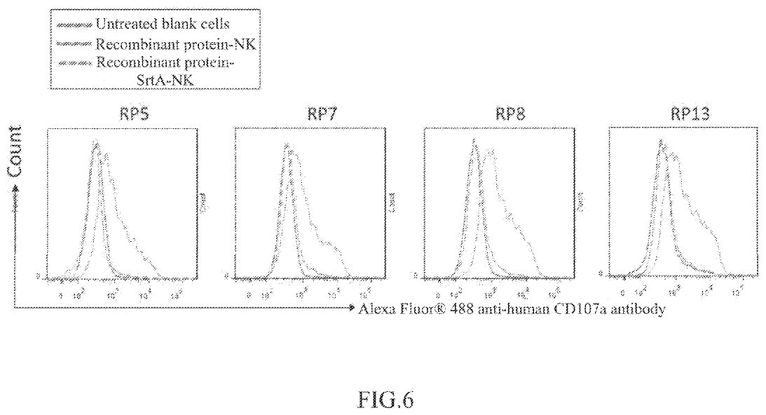

The experimental results are shown in FIG. 6. The results indicate that NK92-FcγRIII cells successfully connected with the Fc segment-containing fusion proteins can be further activated by target cells expressing specific antigens.

IV. NK92-FcγRIII Cells that have been Connected with the Fc Segment-Containing Fusion Proteins can Kill the Target Cells Expressing the Specific Antigens

1. 400 μL of NK92-FcγRIII cell suspension at a concentration of 1.0×10⁶/mL was taken and added with Sortase A to a final concentration of 20 ug/ml and the Fc segment-containing fusion proteins to a final concentration of 10 ug/ml to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-SrtA-NK.

2. 200 μL of NK92-FcγRIII cell suspension at a concentration of 1.0×10⁶/mL was taken and added with the Fc segment-containing fusion proteins alone to a final concentration of 10 ug/ml to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-NK.

3. A 96-well plate was taken and added with 50 μL of suspension of SKOV3 cells (which is an antigen HER2 protein high-expressing cell line) at a concentration of 4.0×10⁵/mL, and then with 50 μL of cell suspension after completion of step 1 or 2 at a concentration of 2×10⁶/mL to obtain an incubation system. The incubation system was cultured at 37° C. for 4 hrs.

4. A 96-well plate was taken and added with 50 μL of suspension of MCF-7 cells (which is an antigen HER2 protein negative cell line) at a concentration of 4.0×10⁵/mL, and then with 50 μL of cell suspension after completion of step 1 or 2 at a concentration of 2×10⁶/mL was added to obtain an incubation system. The incubation system was cultured at 37° C. for 4 hrs.

5. The system after completing steps 3 and 4 was taken, added with 100 ul of working solution for detecting lactate dehydrogenase (LDH) activity (purchased from Dojindo Laboratories Company, Cat. No.: CK12), and developed in the dark for 15 min, followed by addition of 50 ul of termination solution and determination of OD490 nm absorbance.

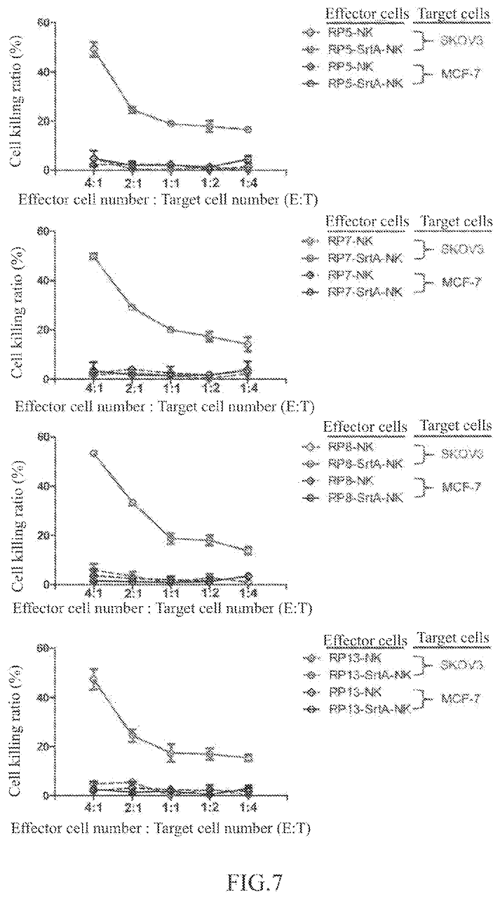

The experimental results are shown in FIG. 7. The results indicate that NK92-FcγRIII cells (RP5-SrtA-NK, RP7-SrtA-NK, RP8-SrtA-NK, RP13-SrtA-NK) that have been connected with the Fc segment-containing fusion proteins can kill SKOV3 cells expressing the specific antigen, but have no killing effect on MCF-7 cells not expressing the antigen. Control NK92-FcγRIII cells (RP5-NK, RP7-NK, RP8-NK, RP13-NK,) that have not been connected with the Fc segment-containing fusion proteins have no killing effect on target cells expressing the specific antigen.

V. Connection of the Fc Segment-Containing Fusion Proteins to Peripheral Blood NK Cells/Peripheral Blood T Cells/Cord Blood NK Cells Using Sortase A

1. Peripheral blood NK cells were purchased from Beijing Junda Kean Technology Co., Ltd.

2. Peripheral blood T cell preparation: mononuclear cells were firstly obtained from human peripheral blood by density gradient centrifugation, and then T cells therein were enriched using the T cell negative selection kit (purchased from Stemcell Company, Cat. No.: 710410) (operated according to the instructions).

3. Cord blood NK cells were donated by Shandong Qilu Stem Cell Engineering Co. LTD.

4. 100 μL of suspension of peripheral blood NK cells or peripheral blood T cells or cord blood NK cells at a concentration of 1.0×10⁶/mL was taken, and added with Sortase A to a final concentration of 20 ug/ml, and Fc segment-containing fusion proteins to a final concentration of 10 ug/ml to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-SrtA-cells.

5. 100 μL of suspension of peripheral blood NK cells or peripheral blood T cells or cord blood NK cells at a concentration of 1.0×10⁶/mL was taken, and added with the Fc segment-containing fusion proteins alone to a final concentration of 10 ug/ml to obtain an incubation system. The incubation system was incubated at 25° C. for 90 min. The cells were collected by centrifugation and washed thoroughly with 0.01 mol/L PBS buffer, pH 7.4. The cells obtained above were named as: recombinant protein-cells.

6. After completing steps 4 and 5, cells were collected by centrifugation, and washed thoroughly with 0.01 mol/L PBS buffer, pH 7.4.

7. The cells collected in step 6 were taken and added with APC-labeled donkey anti-human IgG antibody (APCAffiniPure F(ab′)₂ Fragment Donkey Anti-Human IgG, Fcγ fragment specific, purchased from Jackson ImmunoResearch Company, Cat. No.: 709-136-098) according to the instructions to obtain an incubation system. The incubation system was incubated at 25° C. for 30 min.

8. After completing step 7, cells were collected by centrifugation, and washed thoroughly with 0.01 mol/L PBS buffer, pH 7.4.

9. After completing step 8, the cells without treatment were used as blank group cells, and the cell labeling level was detected using flow cytometry.

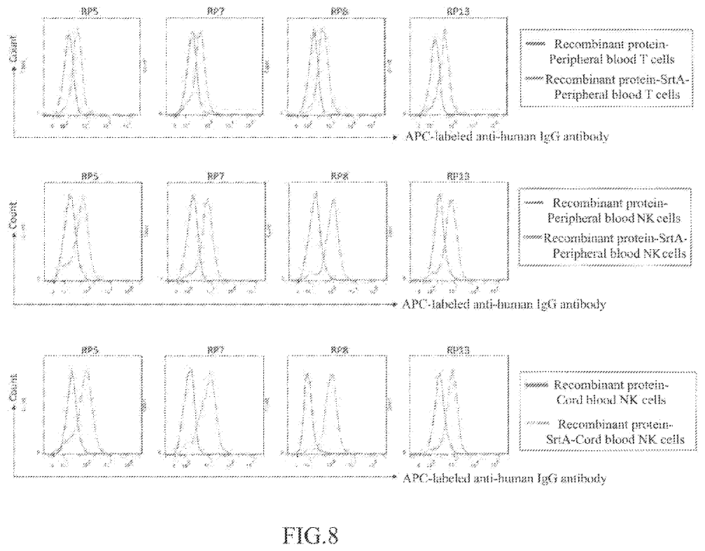

The experimental results are shown in FIG. 8. The results indicate that the Fc segment-containing fusion proteins, which are modified by adding a linking sequence between the Fc segment and the LPXTG region, can be successfully connected directly to the surface of peripheral blood NK cells, peripheral blood T cells and cord blood NK cells mediated by Sortase A.

### CONCLUSION

Our experimental results indicate that the fusion protein obtained through directly fusion and expression of the LPXTG region specifically recognized by Sortase A only at the C-terminus of the Fc segment-containing fusion protein (e.g., intact antibody or Fc fusion protein) (i.e., no linking sequence is added between the Fc segment in the intact antibody or Fc fusion protein and the LPXTG sequence) using the protein modification methods described in the existing patents and literatures (US20160122707A1; Jeong H J, et al., Generation of Ca2+-independent sortase A mutants with enhanced activity for protein and cell surface labeling. PLoS One. 2017 Dec. 4; 12(12):e0189068; Chen I, et al., A general strategy for the evolution of bond-forming enzymes using yeast display. Proc Natl Acad Sci USA. 2011 Jul. 12; 108(28):11399-404), may not be directly connected to the cell surface by Sortase A. We have demonstrated that constructs that connecting the C-terminus of the Fc segment in an intact antibody or Fc fusion protein to the LPXTG region recognized by Sortase A via the specific linking sequences are capable of direct connection to the cell surface mediated by Sortase A. The constructs are RP4, RP5, RP6, RP7, RPB, RP12, RP13, and RP14 as described in the above examples.

1. A fusion protein comprising, from N terminus to C terminus, a first moiety, an Fc segment, a linking moiety, and a substrate moiety of Sortase A, wherein:
the linking moiety comprises a region selected from a linker, and a protein or polypeptide selected from IL2 or scFv, preferably SEQ ID No. 33 or 34;
the linker comprises a sequence selected from the group consisting of:
(1) (GGGGS)n, wherein n≥1 when the linking moiety comprises the protein or polypeptide, and the linker, and n≥3 when the linking moiety comprises only the linker;
(2) (EAAAK)ₙ, n≥1;
the substrate moiety comprises the sequence as shown in LPXTG.

2. The fusion protein according to claim 1, wherein the first moiety is selected from F(ab′), F(ab′)₂, Fab, Fv, scFv, a receptor, and a ligand.

3. The fusion protein according to claim 1, wherein the Fc segment is a wild-type Fc segment or a variant Fc segment,
preferably, the Fc segment is selected from Fc segments of IgG, IgM, IgA, IgD and IgE, preferably from Fc segments of IgG1, IgG2, IgG3 and IgG4.

4. The fusion protein according to claim 1, comprising the structure of any one of:
full-length antibody-GGGGS-GGGGS-GGGGS-LPETGG;
full-length antibody-EAAAK-LPETGG;
full-length antibody-IL2-LPETGG;
full-length antibody-scFv-GGGGS-LPETGG;
full-length antibody-scFv-EAAAK-LPETGG;
scFv-Fc segment-GGGGS-GGGGS-GGGGS-LPETGG; or
scFv-Fc segment-EAAAK-LPETGG.

5. A nucleic acid encoding the fusion protein according to claim 1.

6. The nucleic acid according to claim 5, comprising the encoding sequence of SEQ ID Nos. 4 and 12, SEQ ID Nos. 4 and 14, SEQ ID Nos. 4 and 16, SEQ ID Nos. 4 and 18, SEQ ID Nos. 4 and 20, SEQ ID No. 28, SEQ ID No. 30, or SEQ ID No. 32.

7. A vector comprising the nucleic acid according to claim 6.

8. A host cell comprising the vector according to claim 7.

9. A method of generating the fusion protein, comprising
(1) mixing equimolarly the vector of claim 7 expressing a heavy chain and a vector expressing a light chain in the presence of the light chain;
(2) introducing the vector mixture into a host cell and expressing which for a suitable time under conditions suitable for the expression of the fusion protein; and
(3) recovering the medium supernatant and purifying the fusion protein.

10. A method for connecting the fusion protein according to claim 1 to the surface of a cell, comprising a step of contacting the cell with the fusion protein and Sortase A.

11. The method according to claim 10, wherein the cell is an effector cell, preferably an NK cell or a T cell, preferably a peripheral blood NK cell, a peripheral blood T cell and a cord blood NK cell, preferably an NK92-FcγRIII cell.

12. A cell prepared by the method according to claim 10.

13. A pharmaceutical composition comprising the cell according to claim 12 and a pharmaceutically acceptable carrier.

14. Use of the pharmaceutical composition according to claim 13 in the manufacture of a medicament used for a disease.

15. The use according to claim 14, wherein the disease is a disease caused by abnormal cell proliferation and/or function, preferably a tumor, an autoimmune disease and/or an infectious disease.

16. A method for preventing and/or treating a disease caused by abnormal cell proliferation and/or function, preferably a tumor, an autoimmune disease and/or an infectious disease, comprising the step of providing the composition of claim 13.

17. A vector comprising the nucleic acid according to claim 5.

18. A cell prepared by the method according to claim 11.

19. Use of the cell according to claim 12 in the manufacture of a medicament used for a disease.


SEQUENCE LISTING (34 sequences)

SEQ ID NO: 1 - DNA, 444 bp, Artificial Sequence
  SrtA
atggctaaacctcaaattccgaaagataaatcgaaagtggcaggctatattgaaattccagatgctgatattaaagaaccagtatatccaggaccagcaacaagcgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagaaacgttaagcctacagatgtaggagttctagatgaacaaaaaggtaaagataaacaattaacattaattacttgtgatgattacaatgaaaagacaggcgtttgggaaacccgtaaaatctttgtagctacagaagtcaaataa

SEQ ID NO: 2 - protein, 167 aa, Artificial Sequence
  SrtA
MGSSHHHHHHSSGLVPRGSHMAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTDVGVLDEQKGKDKQLTLITCDDYNEKTGVWETRKIFVATEVK

SEQ ID NO: 3 - DNA, 705 bp, Artificial Sequence
  Ab-CH-LPETGG
atggagaccgacaccctgctgctctgggtgctgctgctctgggtgcccgggtcgaccggtgatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaactcgagatcaaacgtacggtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 4 - protein, 214 aa, Artificial Sequence
  Ab-CH-LPETGG
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1431 bp, Artificial Sequence
  Ab-CH-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaactgcccgagaccggcggctga

SEQ ID NO: 6 - protein, 456 aa, Artificial Sequence
  Ab-CH-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETGG

SEQ ID NO: 7 - DNA, 1446 bp, Artificial Sequence
  Ab-CH-GGGGS-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcctgcccgagaccggcggctga

SEQ ID NO: 8 - protein, 461 aa, Artificial Sequence
  Ab-CH-GGGGS-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLPETGG

SEQ ID NO: 9 - DNA, 1461 bp, Artificial Sequence
  Ab-CH-GGGGS(x2)-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcggcggcggcggcagcctgcccgagaccggcggctga

SEQ ID NO: 10 - protein, 466 aa, Artificial Sequence
  Ab-CH-GGGGS(x2)-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLPETGG

SEQ ID NO: 11 - DNA, 1476 bp, Artificial Sequence
  Ab-CH-GGGGS(x3)-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcggcggcggcggcagcggcggcggcggcagcctgcccgagaccggcggctga

SEQ ID NO: 12 - protein, 471 aa, Artificial Sequence
  Ab-CH-GGGGS(x3)-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLPETGG

SEQ ID NO: 13 - DNA, 1446 bp, Artificial Sequence
  Ab-CH-EAAAK-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagaggccgccgccaagctgcccgagaccggcggctga

SEQ ID NO: 14 - protein, 461 aa, Artificial Sequence
  Ab-CH-EAAAK-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEAAAKLPETGG

SEQ ID NO: 15 - DNA, 1857 bp, Artificial Sequence
  Ab-CH-IL2-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcggatccgcccccacctcctcctccaccaagaagacccagctgcagctggagcacctgctgctggacctgcagatgatcctgaacggcatcaacaactacaagaaccccaagctgaccaggatgctgaccttcaagttctacatgcccaagaaggccaccgagctgaagcacctgcagtgcctggaggaggagctgaagcccctggaggaggtgctgaacctggcccagtccaagaacttccacctgaggcccagggacctgatctccaacatcaacgtgatcgtgctggagctgaagggctccgagaccaccttcatgtgcgagtacgccgacgagaccgccaccatcgtggagttcctgaacaggtggatcaccttctgccagtccatcatctccaccctgacccgtacgctgcccgagaccggcggctga

SEQ ID NO: 16 - protein, 598 aa, Artificial Sequence
  Ab-CH-IL2-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTRTLPETGG

SEQ ID NO: 17 - DNA, 2181 bp, Artificial Sequence
  Ab1-CH-scFv.Ab2-GGGGS-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcgacatccagatgacccagagccccagcagcctgagcgccagcgtgggcgacagggtgaccatcacctgcagggccagccaggacgtgagcaccgccgtggcctggtaccagcagaagcccggcaaggcccccaagctgctgatctacagcgccagcttcctgtacagcggcgtgcccagcaggttcagcggcagcggcagcggcaccgacttcaccctgaccatcagcagcctgcagcccgaggacttcgccacctactactgccagcagtacctgtaccaccccgccaccttcggccagggcaccaaggtggagatcaagggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcgacagctggatccactgggtgaggcaggcccccggcaagggcctggagtgggtggcctggatcagcccctacggcggcagcacctactacgccgacagcgtgaagggcaggttcaccatcagcgccgacaccagcaagaacaccgcctacctgcagatgaacagcctgagggccgaggacaccgccgtgtactactgcgccaggaggcactggcccggcggcttcgactactggggccagggcaccctggtgaccgtgagcagcggcggcggcggcagcctgcccgagaccggcggctga

SEQ ID NO: 18 - protein, 706 aa, Artificial Sequence
  Ab1-CH-scFv.Ab2-GGGGS-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSLPETGG

SEQ ID NO: 19 - DNA, 2181 bp, Artificial Sequence
  Ab1-CH-scFv.Ab2-EAAAK-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcgacatccagatgacccagagccccagcagcctgagcgccagcgtgggcgacagggtgaccatcacctgcagggccagccaggacgtgagcaccgccgtggcctggtaccagcagaagcccggcaaggcccccaagctgctgatctacagcgccagcttcctgtacagcggcgtgcccagcaggttcagcggcagcggcagcggcaccgacttcaccctgaccatcagcagcctgcagcccgaggacttcgccacctactactgccagcagtacctgtaccaccccgccaccttcggccagggcaccaaggtggagatcaagggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcgacagctggatccactgggtgaggcaggcccccggcaagggcctggagtgggtggcctggatcagcccctacggcggcagcacctactacgccgacagcgtgaagggcaggttcaccatcagcgccgacaccagcaagaacaccgcctacctgcagatgaacagcctgagggccgaggacaccgccgtgtactactgcgccaggaggcactggcccggcggcttcgactactggggccagggcaccctggtgaccgtgagcagcgaggccgccgccaagctgcccgagaccggcggctga

SEQ ID NO: 20 - protein, 706 aa, Artificial Sequence
  Ab1-CH-scFv.Ab2-EAAAK-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSEAAAKLPETGG

SEQ ID NO: 21 - DNA, 1509 bp, Artificial Sequence
  scFv-Fc-LPETGG
atggagaccgacaccctgctgctctgggtgctgctgctctgggtgcccgggtcgaccggtgatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaactcgagatcaaaggcggcggcggcagcggcggcggcggcagcggcggcggcggcagcgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaactgcccgagaccggcggctga

SEQ ID NO: 22 - protein, 482 aa, Artificial Sequence
  scFv-Fc-LPETGG
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETGG

SEQ ID NO: 23 - DNA, 1524 bp, Artificial Sequence
  scFv-Fc-GGGGS-LPETGG
atggagaccgacaccctgctgctctgggtgctgctgctctgggtgcccgggtcgaccggtgatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaactcgagatcaaaggcggcggcggcagcggcggcggcggcagcggcggcggcggcagcgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcctgcccgagaccggcggctga

SEQ ID NO: 24 - protein, 487 aa, Artificial Sequence
  scFv-Fc-GGGGS-LPETGG
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLPETGG

SEQ ID NO: 25 - DNA, 1539 bp, Artificial Sequence
  scFv-Fc-(GGGGS)x2-LPETGG
atggagaccgacaccctgctgctctgggtgctgctgctctgggtgcccgggtcgaccggtgatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaactcgagatcaaaggcggcggcggcagcggcggcggcggcagcggcggcggcggcagcgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcggcggcggcggcagcctgcccgagaccggcggctga

SEQ ID NO: 26 - protein, 492 aa, Artificial Sequence
  scFv-Fc-(GGGGS)x2-LPETGG
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLPETGG

SEQ ID NO: 27 - DNA, 1554 bp, Artificial Sequence
  scFv-Fc-(GGGGS)x3-LPETGG
atggagaccgacaccctgctgctctgggtgctgctgctctgggtgcccgggtcgaccggtgatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaactcgagatcaaaggcggcggcggcagcggcggcggcggcagcggcggcggcggcagcgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaaggcggcggcggcagcggcggcggcggcagcggcggcggcggcagcctgcccgagaccggcggctga

SEQ ID NO: 28 - protein, 497 aa, Artificial Sequence
  scFv-Fc-(GGGGS)x3-LPETGG
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLPETGG

SEQ ID NO: 29 - DNA, 1524 bp, Artificial Sequence
  scFv-Fc-EAAAK-LPETGG
atggagaccgacaccctgctgctctgggtgctgctgctctgggtgcccgggtcgaccggtgatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaactcgagatcaaaggcggcggcggcagcggcggcggcggcagcggcggcggcggcagcgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagaggccgccgccaagctgcccgagaccggcggctga

SEQ ID NO: 30 - protein, 487 aa, Artificial Sequence
  scFv-Fc-EAAAK-LPETGG
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEAAAKLPETGG

SEQ ID NO: 31 - DNA, 1437 bp, Artificial Sequence
  Ab(IgG4)-EAAAK-LPETGG
atggagaccgacaccctgctgctctgggtgctgctgctctgggtgcccgggtcgaccggtgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgctagcaccaagggcccatcggtcttccccctggcaccctgctcccgcagtacttctgagagcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcaccaagacctacacgtgcaacgtggatcacaagcccagcaacaccaaggtggacaaacgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagaggccgccgccaagctgcccgagaccggcggctga

SEQ ID NO: 32 - protein, 458 aa, Artificial Sequence
  Ab(IgG4)-EAAAK-LPETGG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEAAAKLPETGG

SEQ ID NO: 33 - protein, 142 aa, Artificial Sequence
  IL2
GGGGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTRT

SEQ ID NO: 34 - protein, 240 aa, Artificial Sequence
  scFv
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS